(19) United States
(12) Patent Application Publication
(10) Pub. No.: US 2007/0213365 A1
(43) Pub. Date: Sep. 13, 2007

(54) Novel polymorphs of montelukast ammonium salts and processes for preparation therefor

(75) Inventors: Itai Adin, Beer-Sheva (IL); Zvicka Deutsch, Kfar-Saba (IL); Michael Brand, RaAnana (IL); Moty Shookrun, Petach-Tikva (IL); Oded Arad, Rechovot (IL); Joseph Kaspi, Givatayim (IL)

Correspondence Address: Martin D. Moynihan;PRTSI, Inc., P.O. Box 16446, Arlington, VA, 22215, US

(73) Assignee: Chemagis Ltd., Bnei-Brak (IL)

(21) Appl. No.: 11/708,612

(22) Filed: Feb. 21, 2007

Related U.S. Application Data
(60) Provisional application No. 60/860,213, filed on Nov. 21, 2006.
(60) Provisional application No. 60/774,647, filed on Feb. 21, 2006.

(51) Int. Cl. A61K 31/47; C07D 215/16

(52) U.S. Cl. 514/311; 546/178

(57) ABSTRACT

Novel crystalline forms I, II, III, IV, V, VI, VII, VIII, IX, and X of montelukast ammonium salts are provided, and novel methods of making these forms are disclosed.

### RELATED APPLICATIONS

The present application claims priority from U.S. Provisional Patent Application No. 60/774,647, filed on Feb. 21, 2006 and U.S. Provisional Patent Application No. 60/860,213, filed on Nov. 21, 2006, which are incorporated herein by reference in their entirety.

### FIELD OF THE INVENTION

The field of the invention relates to solid state chemistry and more particularly to montelukast salts, their properties and preparation processes.

### BACKGROUND OF THE INVENTION

(R-(E)-1-(((1-(3-(2-(7-chloro-2-quinolinyl)ethenyl)phenyl)-3-(2-(1-hydroxy-1-methylethyl)phenyl)propyl)thio)methyl)cyclopropaneacetic acid sodium salt, also known by the name montelukast sodium, is represented by the structural formula I below:

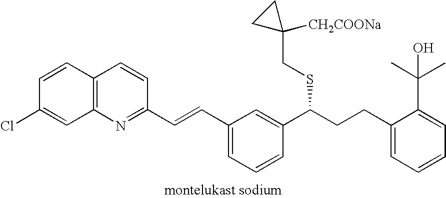

Montelukast sodium is a leukotriene antagonist, and is thus useful as an anti-asthmatic, anti-allergic, anti-inflammatory and cytoprotective agent. Montelukast sodium is currently indicated for the treatment of asthma and allergic rhinitis.

Montelukast sodium is marketed in the United States and other countries by Merck & Co., Inc. under the trade name Singulair®.

Montelukast sodium and related compounds were first disclosed in European Patent No. EP 480,717. The synthesis of montelukast sodium, as taught in patent EP 480,717, involves coupling methyl 1-(mercaptomethyl)cyclopropaneacetate with (S)-1-(3-(2-(7-chloro-2-quinolinyl)ethenyl(phenyl)-3(-2-(1-hydroxy-1-methylethyl)-phenyl)propyl methanesulfonate, followed by hydrolysis of the resulting methyl ester so as to form a free acid, which is converted to the corresponding amorphous sodium salt, obtained by freeze-drying. Both the methyl ester and montelukast acid are not obtained as crystalline materials, as described in example 161, step 6, which refers to example 146 steps 10-12, wherein the montelukast analogue is purified by flash chromatography and accordingly no melting point is mentioned. After the hydrolysis of the ester (step 11), the product is again purified by column chromatography. The data presented in Patent No. EP 480,717 suggests that neither montelukast acid, nor the methyl ester are purified by conventional crystallization and therefore the purification of the resulting montelukast acid is cumbersome.

U.S. Pat. No. 5,523,477 describes the formation of montelukast and the subsequent conversion to the dicyclohexyl ammonium salt, which is converted to montelukast sodium.

U.S. Pat. No. 5,614,632 teaches a method of preparing crystalline montelukast sodium, which involves the preparation of the dilithium dianion of 1-(mercaptomethyl)cyclopropaneacetic acid, followed by condensation thereof with 2-(2-(3-(S)-(3-(7-chloro-2-quinolinyl)ethenyl)phenyl)-3-methanesulfonyloxypropyl)-phenyl)-2-propanol, to yield montelukast acid as a viscous oil. The resulting montelukast acid is converted, via the corresponding dicyclohexyl ammonium salt, to crystalline montelukast sodium. U.S. Pat. No. 5,614,632 refers also to the solid state properties of montelukast acid dicyclohexyl ammonium salt by presenting its X-ray powder diffraction pattern.

The extra purification step via the dicyclohexyl ammonium salt, which is disclosed in U.S. Pat. Nos. 5,523,477 and 5,614,632, is necessitated from the difficulties encountered in purifying montelukast acid. Thus, the crude acid is purified via the dicyclohexylamine salt by reacting it with dicyclohexylamine in ethyl acetate, followed by addition of hexanes to effect crystallization of the dicyclohexylamine salt, or by the crystallization from toluene/heptane. It is mentioned by the inventors of U.S. Pat. No. 5,614,632, that the crystalline montelukast dicyclohexylamine salt offers an efficient method for the purification of montelukast, which circumvents the need to use chromatographic purification.

Patent application US 2005/0107612 describes a process for preparing the t-butyl and phenethyl ammonium salts of montelukast acid in the purification process, as depicted in scheme 1.

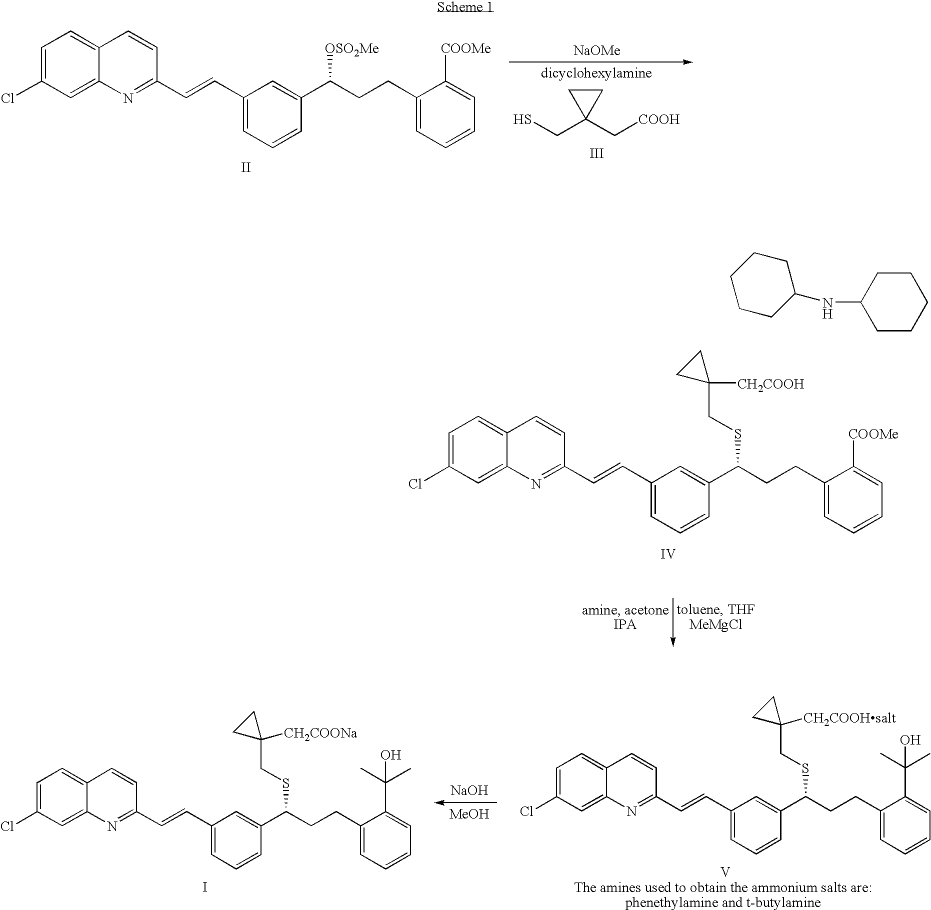

The mesylate intermediate II is converted to the dicyclohexyl ammonium salt of intermediate IV, which is converted to the tert-butyl ammonium salt or to the phenethyl ammonium salt of montelukast acid and then to the corresponding montelukast sodium salt. The calculated yield of the obtained montelukast acid t-butyl ammonium salt in example 6 of the US 2005/0107612 Application is about 62%. The solid state of the tert-butyl ammonium salt or the phenethyl ammonium salt of montelukast acid are not reported.

The use of the tert-butyl ammonium salt of montelukast acid in the preparation of montelukast sodium is recited also in Application WO 2006/043846.

A similar process to the one disclosed in the US 2005/0107612 Application is described in Application WO 2006/008751, which is depicted in scheme 2 below. The intermediate methyl 2-[(3S)-[3-[(2E)-(7-chloro-2-quinolinyl)ethenyl)phenyl]-3-chloropropyl)benzoate (VI) is reacted with 1-(mercaptomethyl)cyclopropaneacetic acid in the presence of sodium hydride to afford the dicyclohexyl ammonium salt of intermediate VII, which is neutralized and converted to montelukast acid or its ammonium salt thereof and then to the corresponding montelukast sodium salt.

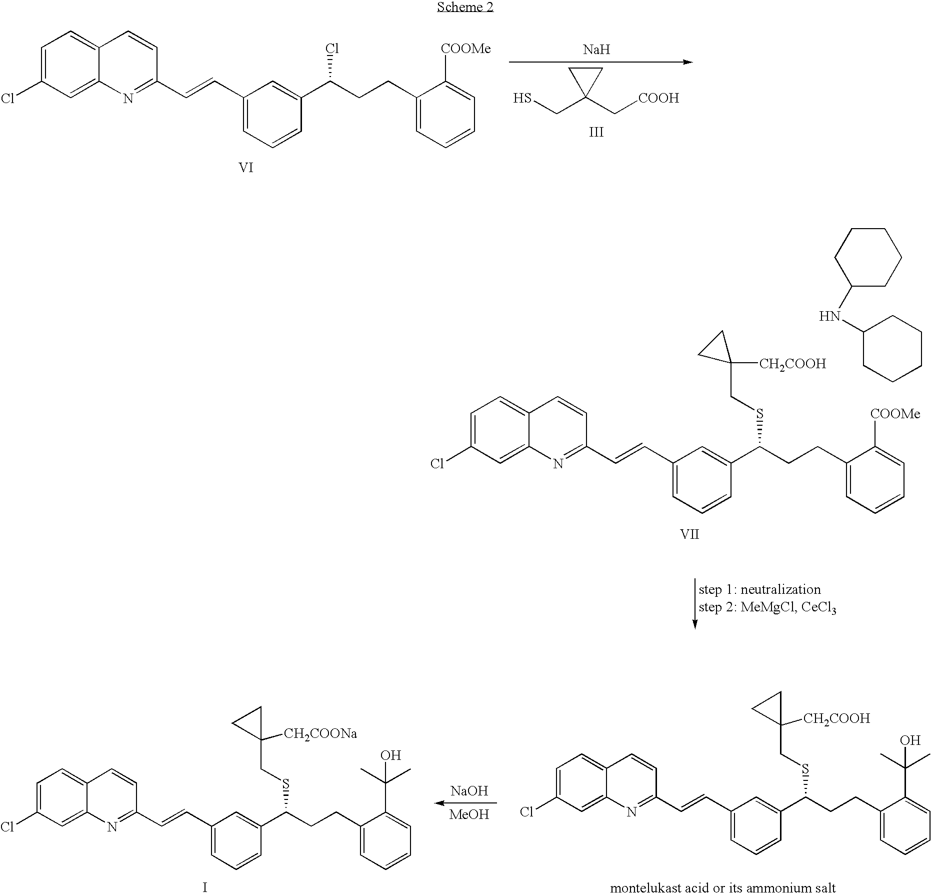

It is stated in the WO 2006/008751 Application that one object of the invention is to provide montelukast organic ammonium salts for the use in preparing montelukast alkali salts. Thus, in this case the solid state characteristics are reported for the dipropyl, α-methylbenzyl, dibenzyl, and diisopropyl ammonium salts. However, the salts are obtained in relatively low yields in the range of 40.5-65%, and only in the case of the dipropylamine salt a yield of 78% is obtained. According to example 7 of U.S. Pat. No. 5,614,632, montelukast dicyclohexyl ammonium salt is obtained in 79%.

Application WO 2007/004237 recites using α-methylbenzyl, dicyclohexyl, and cyclohexylethyl ammonium salts for preparing montelukast sodium.

Application WO 2007/005965 recites using the dipropyl ammonium salt of montelukast acid for preparing purified montelukast sodium.

While it may be inferred from the above detailed description that purification of the crystalline montelukast acid ammonium salt is instrumental in preparing crystalline montelukast sodium, the low yields afforded are a serious drawback for an industrial process. Furthermore, the purification via the dicylohexyl ammonium salt of either montelukast acid or its precursor namely compound VII (see Scheme 2) or compound IV (see Scheme 1) is always part of the process. Hence, there is still a need in the art for a process for preparing other possible montelukast salts in higher yields, which are more economical for industrial use.

### SUMMARY OF THE INVENTION

The present invention provides crystalline solids comprising montelukast acid cyclopentyl ammonium salt, cyclohexyl ammonium salt, cycloheptyl ammonium salt, cyclododecyl ammonium salt, phenethyl ammonium salt, and cyclooctyl ammonium salts.

According to the present invention, each one of the montelukast acid ammonium salts provided herein is a crystalline material, that can be used thereof in a process for preparing highly pure montelukast sodium in high yield.

In one embodiment, the present invention provides a crystalline solid comprising montelukast phenethyl ammonium salt form I. The montelukast phenethyl ammonium salt form I is characterized by unique powder X-ray diffraction pattern (table 1, FIG. 1). The montelukast phenethyl ammonium salt form I, is further characterized by a characteristic IR spectrum as depicted in FIG. 2. The montelukast phenethyl ammonium salt form I is further characterized by characteristic DSC and TGA curves as depicted in FIGS. 3 and 4 respectively.

In another embodiment, the present invention provides a process for preparing the montelukast phenethyl ammonium salt form I by crystallization from ethyl acetate.

In another embodiment, the present invention provides a crystalline solid comprising montelukast cyclohexyl ammonium salt form II, or montelukast cycloheptyl ammonium salt form II. The montelukast cycloheptyl ammonium salt form II is characterized by unique powder X-ray diffraction pattern (table 2, FIG. 5). The montelukast cycloheptyl ammonium salt form II is further characterized by a unique infra-red spectrum, which is depicted in FIG. 6. The montelukast cyclohepyl ammonium salt form II is further characterized by the DSC curve, which is depicted in FIG. 7. The montelukast cyclohexyl ammonium salt form II is characterized by the DSC curve, which is depicted in FIG. 8. The montelukast cyclohexyl ammonium salt form II is further characterized by the thermogravimetric analysis (TGA) curve, which is depicted in FIG. 9.

In yet another embodiment, the present invention provides a process for preparing the montelukast cyclohexyl ammonium salt form II by crystallization from acetonitrile or a mixture of toluene and cyclohexane.

In yet another embodiment, the present invention provides a process for preparing the montelukast cycloheptyl ammonium salt form II by crystallization from acetonitrile, or toluene, or ethyl acetate.

In yet another embodiment, the present invention provides a crystalline solid comprising montelukast cyclopentyl ammonium salt form III. The montelukast cyclopentyl ammonium salt form III is characterized by unique powder X-ray diffraction (table 3, FIG. 10). The cyclopentyl ammonium salt form III, is further characterized by unique infra-red spectrum as depicted in FIG. 11. The montelukast cyclopentyl ammonium salt form III is further characterized by DSC and TGA curves as depicted in FIGS. 12 and 13 respectively.

In yet another embodiment, the present invention provides a process for preparing the crystalline solid comprising montelukast cyclopentyl ammonium salt form III by crystallization from ethyl acetate or toluene.

In yet another embodiment, the present invention provides a crystalline solid comprising montelukast cyclododecyl ammonium salt form IV. The montelukast cyclododecyl ammonium salt form IV is characterized by unique powder X-ray diffraction pattern (table 4, FIG. 14). The montelukast cyclododecyl ammonium salt form IV is further characterized by a unique infra-red spectrum, which is depicted in FIG. 15. The montelukast cyclododecyl ammonium salt form IV is further characterized by DSC and TGA curves, which are depicted in FIGS. 16 and 17 respectively.

In yet another embodiment, the present invention provides a process for preparing the montelukast cyclododecyl ammonium salt form IV by crystallization from ethyl acetate, or acetonitrile, or a mixture of toluene and cyclohexane.

In yet another embodiment, the present invention provides a crystalline solid comprising montelukast cyclohexyl ammonium salt form V. The montelukast cyclohexyl ammonium salt form V is characterized by unique powder X-ray diffraction pattern (table 5, FIG. 18). The montelukast cyclohexyl ammonium salt form V is further characterized by a unique infra-red spectrum, which is depicted in FIG. 19. The montelukast cyclohexyl ammonium salt form V is further characterized by DSC and TGA curves, which are depicted in FIGS. 20 and 21 respectively.

In yet another embodiment, the present invention provides a process for preparing the crystalline solid comprising montelukast cyclohexyl ammonium salt form V by crystallization from a mixture of ethyl acetate and cyclohexane.

In yet another embodiment, the present invention provides a crystalline solid comprising montelukast phenethyl ammonium salt form VI. The montelukast phenethyl ammonium salt form VI is characterized by unique powder X-ray diffraction pattern (table 6, FIG. 22). The montelukast phenethyl ammonium salt form VI is further characterized by a unique infra-red spectrum, which is depicted in FIG. 23. The montelukast phenethyl ammonium salt form VI is further characterized by DSC and TGA curves, which are depicted in FIGS. 24 and 25 respectively.

In yet another embodiment, the present invention provides a process for preparing the montelukast phenethyl ammonium salt form VI by crystallization from a mixture of ethyl acetate and cyclohexane.

In yet another embodiment, the present invention provides a crystalline solid comprising montelukast cyclopentyl ammonium salt form VII. The montelukast cyclopentyl ammonium salt form VII is characterized by unique powder X-ray diffraction pattern (table 7, FIG. 26). The montelukast cyclopentyl ammonium salt form VII is further characterized by a unique infra-red spectrum, which is depicted in FIG. 27. The montelukast cyclopentyl ammonium salt form VII is further characterized by DSC and TGA curves, which are depicted in FIGS. 28 and 29 respectively.

In yet another embodiment, the present invention provides a process for preparing the crystalline solid comprising montelukast cyclopentyl ammonium salt form VII by crystallization from acetonitrile.

In yet another embodiment, the present invention provides a crystalline solid comprising montelukast cyclooctyl ammonium salt form VIII. The montelukast cyclooctyl ammonium salt form VIII is characterized by unique powder X-ray diffraction pattern (table 8, FIG. 30).

The montelukast cyclooctyl ammonium salt form VIII is further characterized by a unique infra-red spectrum, which is depicted in FIG. 31. The montelukast cyclooctyl ammonium salt form VIII is further characterized by DSC and TGA curves, which are depicted in FIGS. 32 and 33 respectively.

In yet another embodiment, the present invention provides a process for preparing the crystalline solid comprising montelukast cyclooctyl ammonium salt form VIII, the process comprising:
providing a mixture of montelukast acid in an organic solvent while stirring and optionally heating to elevated temperature;
adding cyclooctylamine and optionally cooling to obtain a suspension;
stirring for sufficient time to allow crystallization;
obtaining the crystals by filtering and washing; and
optionally drying the obtained crystals.

Preferably, the organic solvent is selected from the group consisting of toluene, ethyl acetate, and acetonitrile.

In yet another embodiment, the present invention provides another process for preparing the crystalline solid comprising montelukast cyclooctyl ammonium salt form VIII, the process comprising:
providing a mixture of montelukast cyclooctyl ammonium salt in an organic solvent while stirring and heating to elevated temperature;
stirring for sufficient time to allow crystallization;
obtaining the crystals by filtering and washing; and
optionally drying the obtained crystals.

In yet another embodiment, the present invention provides yet another process for preparing the crystalline solid comprising montelukast cyclooctyl ammonium salt form VIII, which process comprises:
providing a mixture of montelukast cyclooctyl ammonium salt in an organic solvent while stirring and heating to elevated temperature;
adding an anti-solvent upon cooling;
stirring for sufficient time to allow crystallization;
obtaining the crystals by filtering and washing; and
optionally drying the obtained crystals.

In yet another embodiment, the present invention provides a crystalline solid comprising montelukast cyclooctyl ammonium salt form IX. The montelukast cyclooctyl ammonium salt form IX is characterized by unique powder X-ray diffraction pattern (table 9, FIG. 34). The montelukast cyclooctyl ammonium salt form IX is further characterized by a unique infra-red spectrum, which is depicted in FIG. 35. The montelukast cyclooctyl ammonium salt form IX is further characterized by DSC and TGA curves, which are depicted in FIGS. 36 and 37 respectively.

In yet another embodiment, the present invention provides a process for preparing the crystalline solid comprising montelukast cyclooctyl ammonium salt form IX, the process comprising:
providing a mixture of montelukast cyclooctyl ammonium salt in nitroethane while stirring and heating to elevated temperature;
stirring for sufficient time to allow crystallization;
obtaining the crystals by filtering and washing; and
optionally drying the obtained crystals.

Another process of the present invention for preparing the crystalline solid comprising montelukast cyclooctyl ammonium salt form IX, comprises:
providing a mixture of montelukast cyclooctyl ammonium salt in an organic solvent while stirring and heating to elevated temperature;
adding an anti-solvent upon cooling;
stirring for sufficient time to allow crystallization;
obtaining the crystals by filtering and washing; and
optionally drying the obtained crystals.

In yet another embodiment, the present invention provides a crystalline solid comprising montelukast cyclooctyl ammonium salt form X. The montelukast cyclooctyl ammonium salt form X is characterized by unique powder X-ray diffraction pattern (table 10, FIG. 38). The montelukast cyclooctyl ammonium salt form X is further characterized by a unique infra-red spectrum, which is depicted in FIG. 39. The montelukast cyclooctyl ammonium salt form X is further characterized by DSC and TGA curves, which are depicted in FIGS. 40 and 41 respectively.

In yet another embodiment, the present invention provides a process for preparing the crystalline solid comprising montelukast cyclooctyl ammonium salt form X, the process comprising:
providing a mixture of montelukast cyclooctyl ammonium salt in acetonitrile while stirring and heating to elevated temperature;
rapidly cooling the mixture while maintaining stirring;
obtaining the crystals by filtering and washing; and
optionally drying the obtained crystals.

Another process of the present invention for preparing the crystalline solid comprising montelukast cyclooctyl ammonium salt form X, comprises:
providing a mixture of montelukast cyclooctyl ammonium salt in an organic solvent while stirring and optionally heating to elevated temperature;
adding an anti-solvent upon cooling;
stirring for sufficient time to allow crystallization;
obtaining the crystals by filtering and washing; and
optionally drying the obtained crystals.

In yet another embodiment, the present invention provides amorphous montelukast cyclooctyl ammonium salt.

### BRIEF DESCRIPTION OF THE FIGURES

FIG. 1 depicts the powder X-ray diffraction pattern of montelukast phenethyl ammonium salt form I.

FIG. 2 depicts the infra-red spectrum of montelukast phenethyl ammonium salt form I.

FIG. 3 depicts the differential scanning calorimetry (DSC) curve of montelukast phenethyl ammonium salt form I.

FIG. 4 depicts the thermogravimetric analysis (TGA) curve of montelukast phenethyl ammonium salt form I.

FIG. 5 depicts the powder X-ray diffraction pattern of the montelukast cyclohepyl ammonium salt form II.

FIG. 6 depicts the infra-red spectrum of the montelukast cyclohepyl ammonium salt form II.

FIG. 7 depicts the DSC curve of the montelukast cyclohepyl ammonium salt form II.

FIG. 8 depicts the DSC curve of the montelukast cyclohexyl ammonium salt form II.

FIG. 9 depicts the thermogravimetric analysis (TGA) curve the montelukast cyclohexyl ammonium salt form II.

FIG. 10 depicts the powder X-ray diffraction pattern of montelukast cyclopentyl ammonium salt form III.

FIG. 11 depicts the infra-red spectrum of montelukast cyclopentyl ammonium salt form III.

FIG. 12 depicts the differential scanning calorimetry (DSC) curve of montelukast cyclopentyl ammonium salt form III.

FIG. 13 depicts the thermogravimetric analysis (TGA) curve of montelukast cyclopentyl ammonium salt form III.

FIG. 14 depicts the powder X-ray diffraction pattern of montelukast cyclododecyl ammonium salt form IV.

FIG. 15 depicts the infra-red spectrum of montelukast cyclododecyl ammonium salt form IV.

FIG. 16 depicts the differential scanning calorimetry (DSC) curve of montelukast cyclododecyl ammonium salt form IV.

FIG. 17 depicts the thermogravimetric analysis (TGA) curve of montelukast cyclododecyl ammonium salt form IV.

FIG. 18 depicts the powder X-ray diffraction pattern of montelukast cyclohexyl ammonium salt form V.

FIG. 19 depicts the infra-red spectrum of montelukast cyclohexyl ammonium salt form V.

FIG. 20 depicts the differential scanning calorimetry (DSC) curve of montelukast cyclohexyl ammonium salt form V.

FIG. 21 depicts the thermogravimetric analysis (TGA) curve of montelukast cyclohexyl ammonium salt form V.

FIG. 22 depicts the powder X-ray diffraction pattern of montelukast phenethyl ammonium salt form VI.

FIG. 23 depicts the infra-red spectrum of montelukast phenethyl ammonium salt form VI.

FIG. 24 depicts the differential scanning calorimetry (DSC) curve of montelukast phenethyl ammonium salt form VI.

FIG. 25 depicts the thermogravimetric analysis (TGA) curve of montelukast phenethyl ammonium salt form VI.

FIG. 26 depicts the powder X-ray diffraction pattern of montelukast cyclopentyl ammonium salt form VII.

FIG. 27 depicts the infra-red spectrum of montelukast cyclopentyl ammonium salt form VII.

FIG. 28 depicts the differential scanning calorimetry (DSC) curve of montelukast cyclopentyl ammonium salt form VII.

FIG. 29 depicts the thermogravimetric analysis (TGA) curve of montelukast cyclopentyl ammonium salt form VII.

FIG. 30 depicts the powder X-ray diffraction pattern of montelukast cyclooctyl ammonium salt form VIII.

FIG. 31 depicts the infra-red spectrum of montelukast cyclooctyl ammonium salt form VIII.

FIG. 32 depicts the differential scanning calorimetry (DSC) curve of montelukast cyclooctyl ammonium salt form VIII.

FIG. 33 depicts the thermogravimetric analysis (TGA) curve of montelukast cyclooctyl ammonium salt form VIII.

FIG. 34 depicts the powder X-ray diffraction pattern of montelukast cyclooctyl ammonium salt form IX.

FIG. 35 depicts the infra-red spectrum of montelukast cyclooctyl ammonium salt form IX.

FIG. 36 depicts the differential scanning calorimetry (DSC) curve of montelukast cyclooctyl ammonium salt form IX.

FIG. 37 depicts the thermogravimetric analysis (TGA) curve of montelukast cyclooctyl ammonium salt form IX.

FIG. 38 depicts the powder X-ray diffraction pattern of montelukast cyclooctyl ammonium salt form X.

FIG. 39 depicts the infra-red spectrum of montelukast cyclooctyl ammonium salt form X.

FIG. 40 depicts the differential scanning calorimetry (DSC) curve of montelukast cyclooctyl ammonium salt form X.

FIG. 41 depicts the thermogravimetric analysis (TGA) curve of montelukast cyclooctyl ammonium salt form X.

### DESCRIPTION OF THE PREFERRED EMBODIMENTS

The present inventors have surprisingly uncovered new salt forms of (R-(E)-1-(((1-(3-(2-(7-chloro-2-quinolinyl)ethenyl)phenyl)-3-(2-(1-hydroxy-1-methylethyl)-phenyl)propyl)thio)methyl)cyclopropaneacetic acid, also known by the name montelukast acid. The said salt forms are obtained in high yields and are characterized by improved properties.

The salts can be prepared by any method known in the art for preparing addition salts of active pharmaceutical ingredients e.g., by treating the active pharmaceutical ingredient (e.g., montelukast acid, obtained by any method known in the art) with a base (e.g., an organic amine) to obtain its salt form, i.e., the montelukast ammonium salt, or by other methods, as demonstrated e.g., by example 1.

Thus, the present invention provides crystalline solids comprising montelukast acid cyclopentyl ammonium salt, cyclohexyl ammonium salt, cycloheptyl ammonium salt, cyclododecyl ammonium salt, phenethyl ammonium salt, and cyclooctyl ammonium salts.

According to the present invention, each one of the montelukast acid ammonium salts provided herein is a crystalline material, that can be used thereof in a process for preparing highly pure montelukast sodium. Thus, the present invention further provides a process for preparing montelukast sodium from a crystalline montelukast ammonium salt. The process preferably includes converting the crystalline montelukast ammonium salt to montelukast sodium.

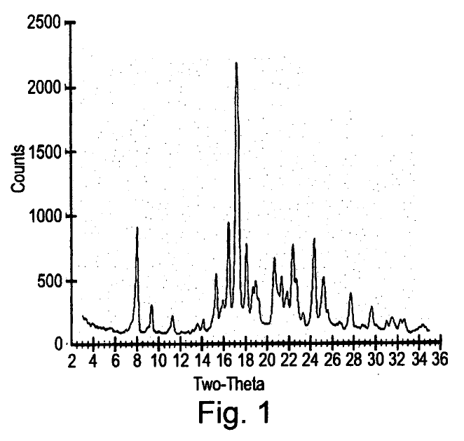

In one embodiment, the present invention provides a crystalline solid comprising montelukast phenethyl ammonium salt form I. The montelukast phenethyl ammonium salt form I is characterized by unique powder X-ray diffraction pattern (table 1, FIG. 1). The strong diffraction peaks at 8.0, 15.3, 16.5, 17.3, 18.1, 20.7, 21.3, 22.4, 24.4, and 25.2±0.2 degrees 2θ are most characteristic of this form.

TABLE 1
montelukast phenethyl ammonium salt form I - Powder
X-ray diffraction peak positions and intensities

| 2θ degrees | I/I0 |
|---|---|
| 8.0 | 43.6 |
| 9.4 | 11.4 |
| 11.3 | 7.8 |
| 13.6 | 3.5 |
| 14.1 | 5.2 |
| 15.3 | 21.9 |
| 15.9 | 12.2 |
| 16.5 | 43.2 |
| 17.3 | 100.0 |
| 18.1 | 33.0 |
| 18.7 | 15.2 |
| 19.0 | 18.4 |
| 19.2 | 10.7 |
| 20.7 | 27.7 |
| 21.3 | 20.8 |
| 21.9 | 14.3 |
| 22.4 | 32.4 |
| 22.7 | 18.6 |
| 23.3 | 6.8 |
| 24.4 | 35.8 |
| 25.2 | 20.4 |
| 25.6 | 6.7 |
| 26.7 | 3.3 |
| 27.7 | 14.2 |
| 28.8 | 2.2 |
| 29.6 | 9.1 |
| 31.0 | 4.3 |
| 31.5 | 5.4 |
| 32.3 | 3.8 |
| 32.6 | 4.3 |
| 34.3 | 2.7 |

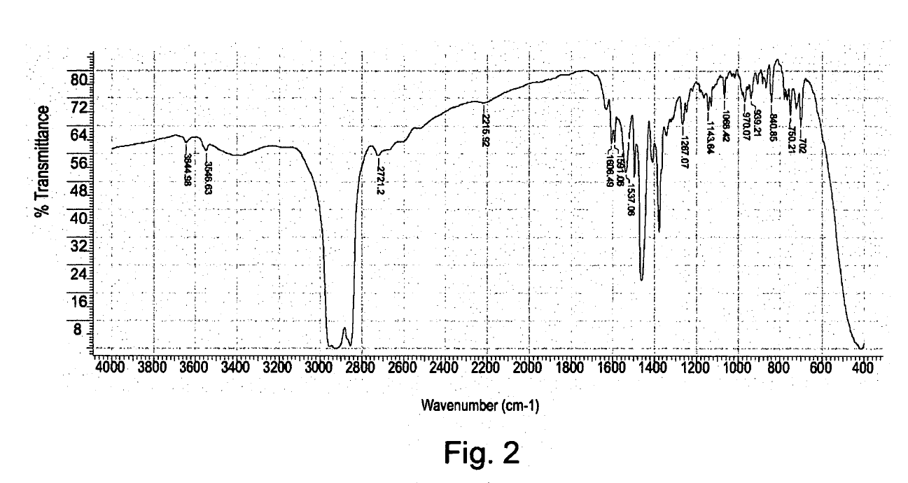

The montelukast phenethyl ammonium salt form I, is further characterized by a characteristic IR spectrum as depicted in FIG. 2.

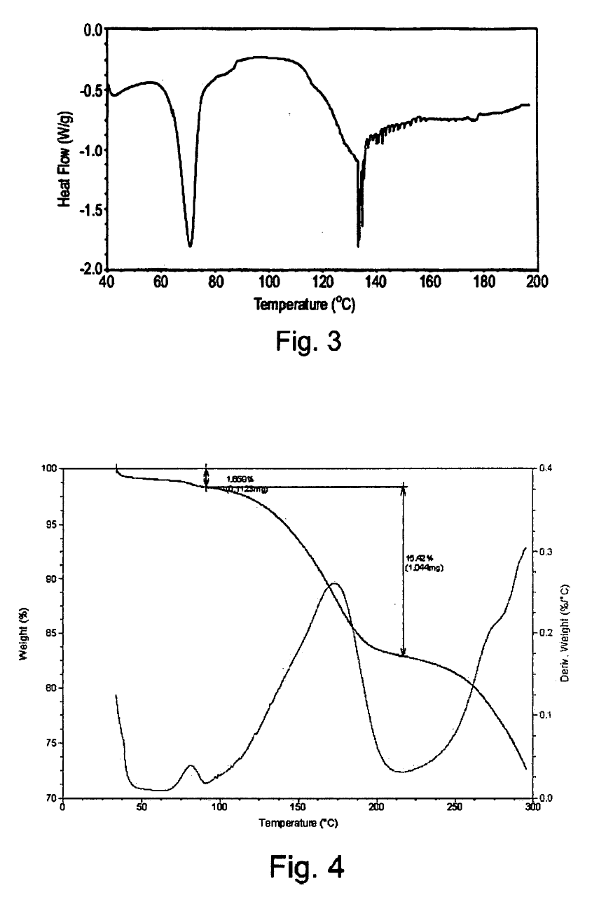

The montelukast phenethyl ammonium salt form I is further characterized by characteristic DSC and TGA curves as depicted in FIGS. 3 and 4 respectively.

In another embodiment, the present invention provides a process for preparing the crystalline solid comprising montelukast phenethyl ammonium salt form I, the process comprising:
providing a mixture of montelukast acid in ethyl acetate while stirring and optionally heating to elevated temperature;
adding phenethylamine and optionally cooling to obtain a suspension;
stirring for sufficient time to allow crystallization;
obtaining the crystals by filtering and washing; and
optionally drying the obtained crystals.

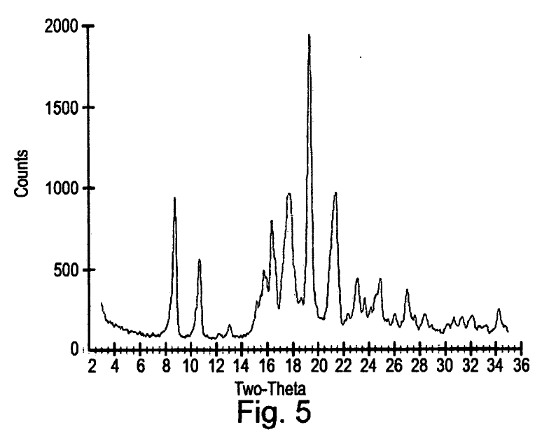

In another embodiment, the present invention provides a crystalline solid comprising montelukast cyclohexyl ammonium salt form II, or montelukast cycloheptyl ammonium salt form II. The montelukast cycloheptyl ammonium salt form II is characterized by unique powder X-ray diffraction pattern (table 2, FIG. 5). The diffraction peaks at 8.8, 10.7, 15.7, 16.4, 16.6, 17.7, 19.4, and 21.4±0.2 degrees 2θ are most characteristic of this form.

TABLE 2
montelukast cycloheptyl ammonium salt form II - Powder X-ray
diffraction peak positions and intensities

| 2θ degrees | I/I0 |
|---|---|
| 8.8 | 53.4 |
| 10.7 | 28.2 |
| 12.1 | 2.0 |
| 13.0 | 4.4 |
| 15.3 | 12.1 |
| 15.7 | 23.1 |
| 16.4 | 39.9 |
| 16.6 | 23.3 |
| 17.7 | 49.8 |
| 19.4 | 100.0 |
| 21.4 | 49.3 |
| 23.1 | 18.7 |
| 23.6 | 9.9 |
| 24.1 | 10.7 |
| 24.5 | 12.7 |
| 24.9 | 18.0 |
| 26.1 | 5.9 |
| 27.0 | 13.4 |
| 27.6 | 6.5 |
| 28.4 | 8.2 |
| 30.1 | 3.8 |
| 30.6 | 5.2 |
| 31.3 | 6.8 |
| 32.1 | 6.3 |
| 33.2 | 3.7 |
| 34.3 | 8.1 |

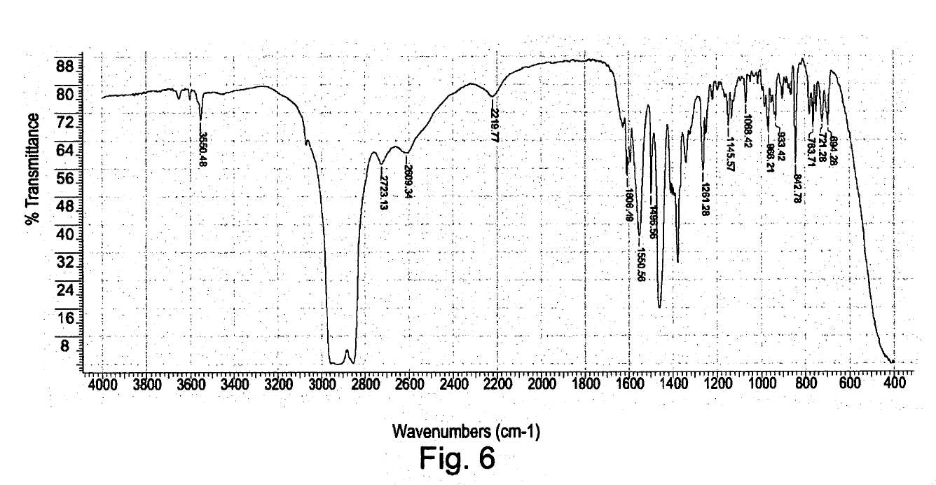

The montelukast cycloheptyl ammonium salt form II is further characterized by a unique infra-red spectrum, which is depicted in FIG. 6.

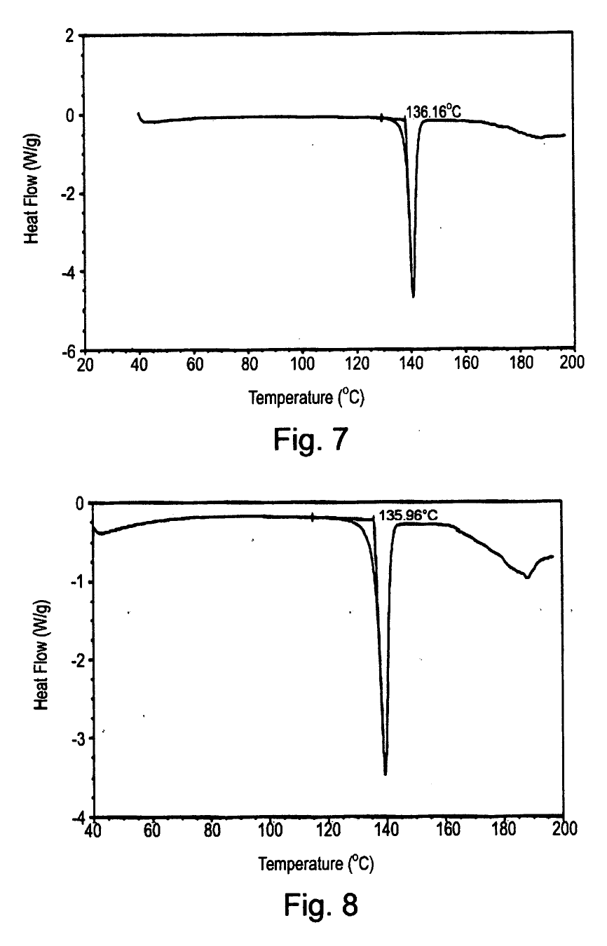

The montelukast cyclohepyl ammonium salt form II is further characterized by a DSC curve, which is depicted in FIG. 7.

The montelukast cyclohexyl ammonium salt form II is characterized by a DSC curve, which is depicted in FIG. 8.

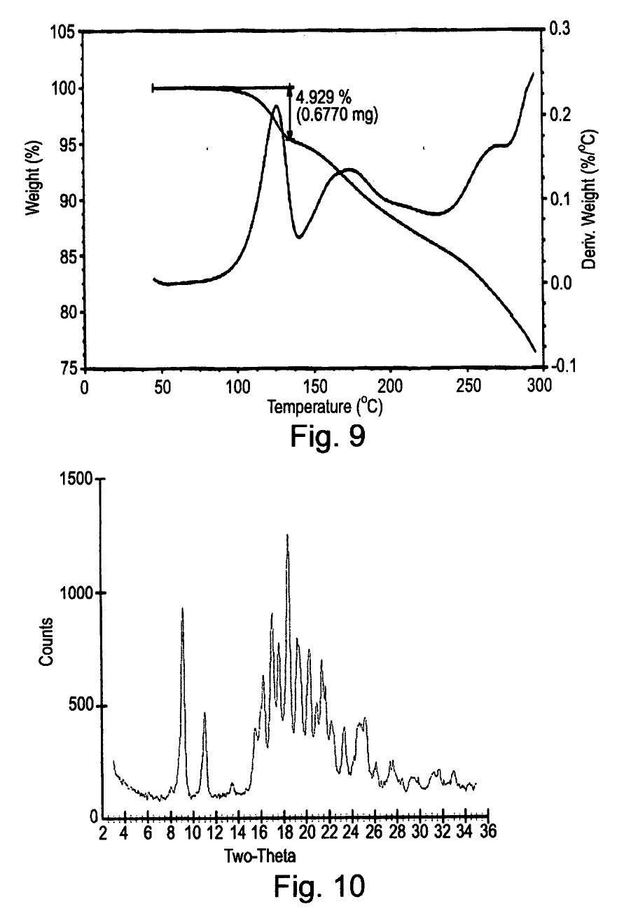

The montelukast cyclohexyl ammonium salt form II is further characterized by a thermogravimetric analysis (TGA) curve, which is depicted in FIG. 9.

In yet another embodiment, the present invention provides a process for preparing the crystalline solid comprising montelukast cyclohexyl ammonium salt form II, the process comprising:
providing a mixture of montelukast acid in an organic solvent while stirring and optionally heating to elevated temperature;
adding cyclohexylamine and optionally cooling to obtain a suspension;
stirring for sufficient time to allow crystallization;
obtaining the crystals by filtering and washing; and
optionally drying the obtained crystals.

Preferably, the organic solvent is selected from the group consisting of cyclohexane, toluene, acetonitrile, and mixtures thereof.

In yet another embodiment, the present invention provides a process for preparing the crystalline solid comprising montelukast cycloheptyl ammonium salt form II, the process comprising:
providing a mixture of montelukast acid in an organic solvent while stirring and optionally heating to elevated temperature;
adding cycloheptylamine and optionally cooling to obtain a suspension;
stirring for sufficient time to allow crystallization;
obtaining the crystals by filtering and washing; and
optionally drying the obtained crystals.

Preferably, the organic solvent is selected from the group consisting of toluene, ethyl acetate, acetonitrile, and mixtures thereof.

In yet another embodiment, the present invention provides a crystalline solid comprising montelukast cyclopentyl ammonium salt form III. The montelukast cyclopentyl ammonium salt form III is characterized by unique powder X-ray diffraction (table 3, FIG. 10). The diffraction peaks at 9.2, 11.1, 15.5, 16.0, 16.2, 17.0, 17.6, 18.5, 19.3, 20.3, 20.9, 21.4, 21.7, 22.2, 23.3, 24.7, and 25.2±0.2 degrees 2θ are most characteristic of this form.

TABLE 3
montelukast cyclopentyl ammonium salt form III - Powder X-ray
diffraction peak positions and intensities

| 2θ degrees | I/I0 |
|---|---|
| 8.0 | 4.6 |
| 9.2 | 77.1 |
| 11.1 | 34.1 |
| 13.4 | 5.8 |
| 15.5 | 27.4 |
| 16.0 | 32.7 |
| 16.2 | 46.7 |
| 17.0 | 71.8 |
| 17.6 | 58.4 |
| 18.5 | 100.0 |
| 19.3 | 61.7 |
| 20.3 | 55.1 |
| 20.9 | 34.7 |
| 21.4 | 52.3 |
| 21.7 | 41.3 |
| 22.2 | 27.0 |
| 23.3 | 24.9 |
| 24.7 | 26.3 |
| 25.2 | 28.3 |
| 26.1 | 11.5 |
| 27.4 | 12.4 |
| 27.6 | 12.6 |
| 28.4 | 5.2 |
| 29.2 | 5.9 |
| 31.3 | 7.8 |
| 31.7 | 9.3 |
| 33.0 | 8.6 |

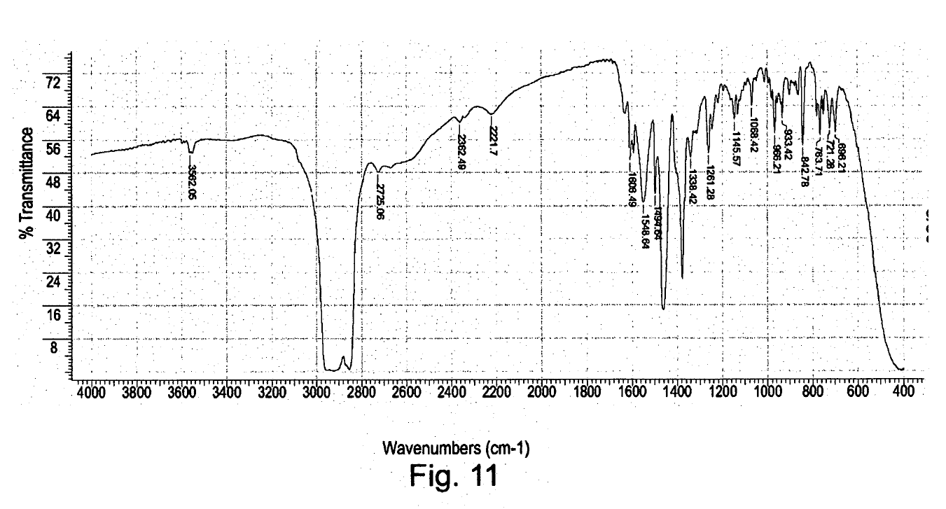

The montelukast cyclopentyl ammonium salt form III is further characterized by unique infra-red spectrum as depicted in FIG. 11.

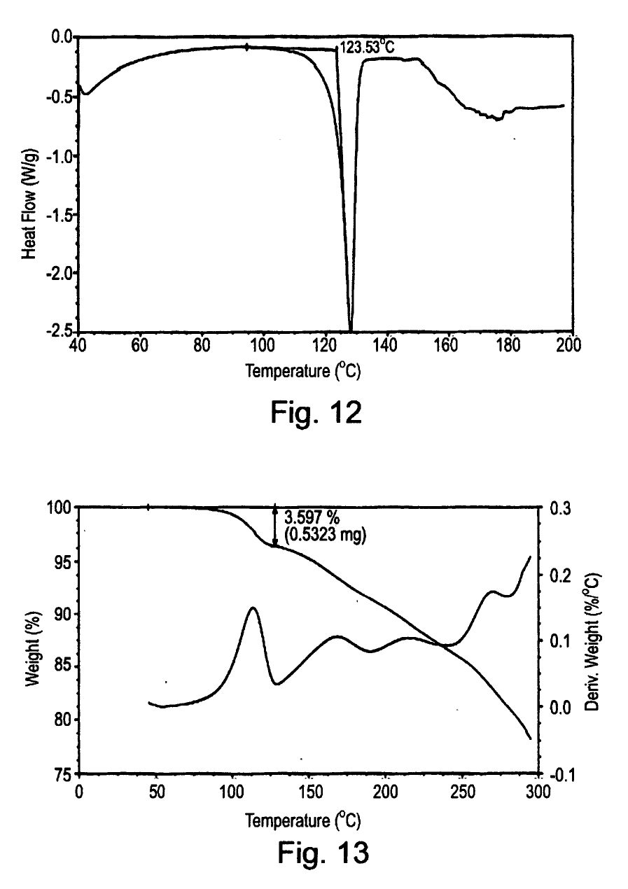

The montelukast cyclopentyl ammonium salt form III is further characterized by DSC and TGA curves as depicted in FIGS. 12 and 13 respectively.

In yet another embodiment, the present invention provides a process for preparing the crystalline solid comprising montelukast cyclopentyl ammonium salt form III, the process comprising:
providing a mixture of montelukast acid in an organic solvent while stirring and optionally heating to elevated temperature;
adding cyclopentylamine and optionally cooling to obtain a suspension;
stirring for sufficient time to allow crystallization;
obtaining the crystals by filtering and washing; and
optionally drying the obtained crystals.

Preferably, the organic solvent is selected from the group consisting of toluene and ethyl acetate.

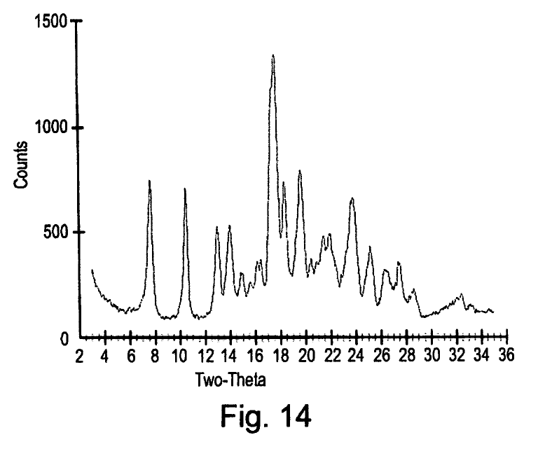

In yet another embodiment, the present invention provides a crystalline solid comprising montelukast cyclododecyl ammonium salt form IV. The montelukast cyclododecyl ammonium salt form IV is characterized by unique powder X-ray diffraction pattern (table 4, FIG. 14). The diffraction peaks at 7.7, 10.5, 13.0, 14.0, 17.7, 18.4, 19.7, 21.5, 21.9, 23.8, 25.2 and 27.4 2±0.2 degrees 2θ are most characteristic of this form.

TABLE 4
montelukast cyclododecyl ammonium salt form IV - Powder
X-ray diffraction peak positions and intensities

| 2θ degrees | I/I0 |
|---|---|
| 7.7 | 56.1 |
| 10.5 | 54.3 |
| 13.0 | 36.0 |
| 14.0 | 35.6 |
| 14.9 | 16.2 |
| 15.6 | 10.2 |
| 16.4 | 19.6 |
| 17.7 | 100.0 |
| 18.4 | 50.0 |
| 19.7 | 53.9 |
| 20.5 | 17.7 |
| 21.5 | 26.3 |
| 21.9 | 27.4 |
| 23.8 | 42.0 |
| 25.2 | 24.9 |
| 26.5 | 15.6 |
| 27.4 | 20.7 |
| 28.7 | 10.5 |
| 32.4 | 8.7 |
| 33.1 | 4.8 |
| 34.6 | 2.9 |

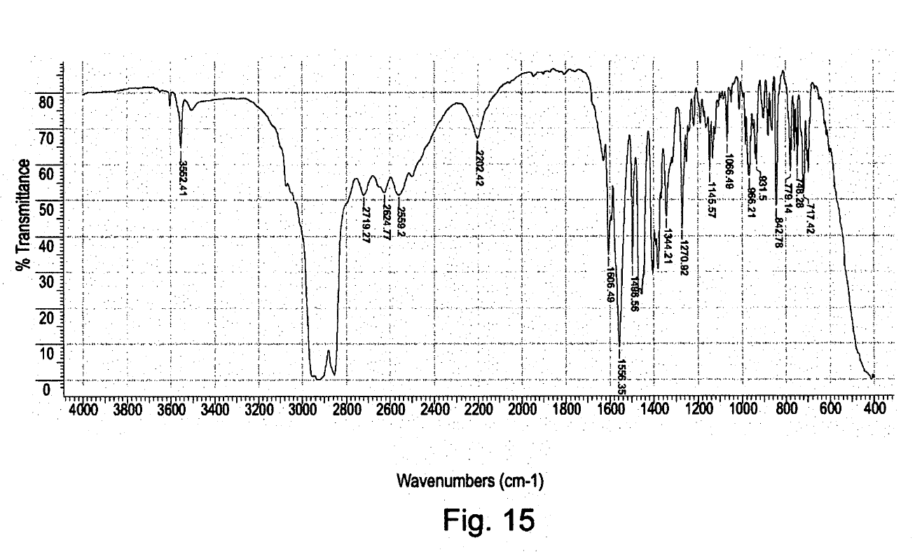

The montelukast cyclododecyl ammonium salt form IV is further characterized by a unique infra-red spectrum, which is depicted in FIG. 15.

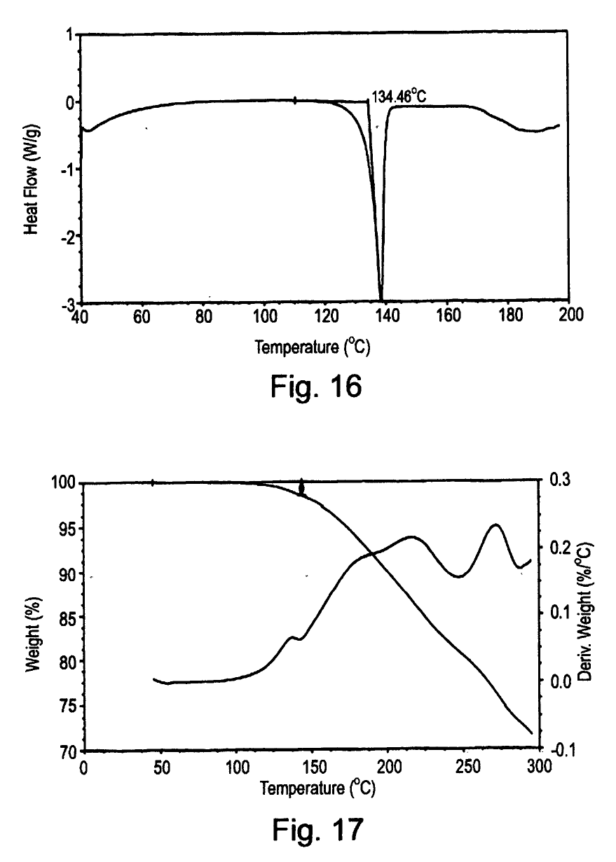

The montelukast cyclododecyl ammonium salt form IV is further characterized by DSC and TGA curves, which are depicted in FIGS. 16 and 17 respectively.

In yet another embodiment, the present invention provides a process for preparing the crystalline solid comprising montelukast cyclododecyl ammonium salt form IV, the process comprising:
providing a mixture of montelukast acid in an organic solvent while stirring and optionally heating to elevated temperature;
adding cyclododecylamine and optionally cooling to obtain a suspension;
stirring for sufficient time to allow crystallization;
obtaining the crystals by filtering and washing; and
optionally drying the obtained crystals.

Preferably, the organic solvent is selected from the group consisting of cyclohexane, toluene, ethyl acetate, acetonitrile, and mixtures thereof.

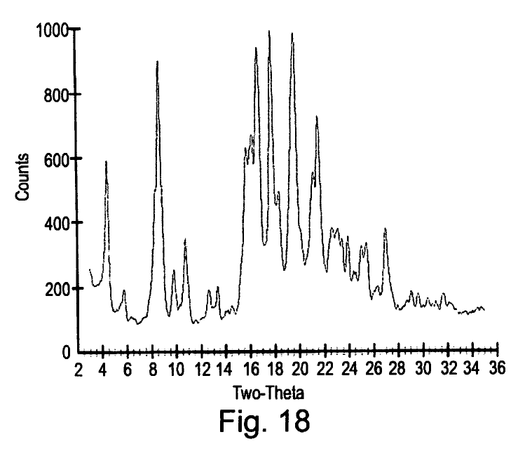

In yet another embodiment, the present invention provides a crystalline solid comprising montelukast cyclohexyl ammonium salt form V. The montelukast cyclohexyl ammonium salt form V is characterized by unique powder X-ray diffraction pattern (table 5, FIG. 18). The diffraction peaks at 4.5, 8.3, 8.7, 9.8, 10.8, 15.7, 16.2, 16.7, 17.8, 18.4, 19.7, 21.2, 21.5, 22.6, 23.1, 23.4, 24.0, 25.5, and 27.0±0.2 degrees 2θ are most characteristic of this form.

TABLE 5
montelukast cyclohexyl ammonium salt form V - Powder X-ray
diffraction peak positions and intensities

| 2θ degrees | I/I0 |
|---|---|
| 4.5 | 61.3 |
| 5.8 | 12.6 |
| 8.3 | 50.1 |
| 8.7 | 100.0 |
| 9.8 | 21.9 |
| 10.8 | 33.6 |
| 12.7 | 11.1 |
| 13.4 | 11.3 |
| 15.7 | 57.4 |
| 16.2 | 60.1 |
| 16.7 | 90.8 |
| 17.8 | 99.2 |
| 18.4 | 40.3 |
| 19.7 | 92.5 |
| 21.2 | 42.0 |
| 21.5 | 60.7 |
| 22.6 | 21.2 |
| 23.1 | 21.5 |
| 23.4 | 20.2 |
| 24.0 | 21.7 |
| 24.6 | 10.8 |
| 25.0 | 18.8 |
| 25.5 | 21.1 |
| 26.4 | 7.1 |
| 27.0 | 28.8 |
| 29.1 | 8.1 |
| 29.6 | 6.6 |
| 30.4 | 5.7 |
| 31.7 | 6.7 |

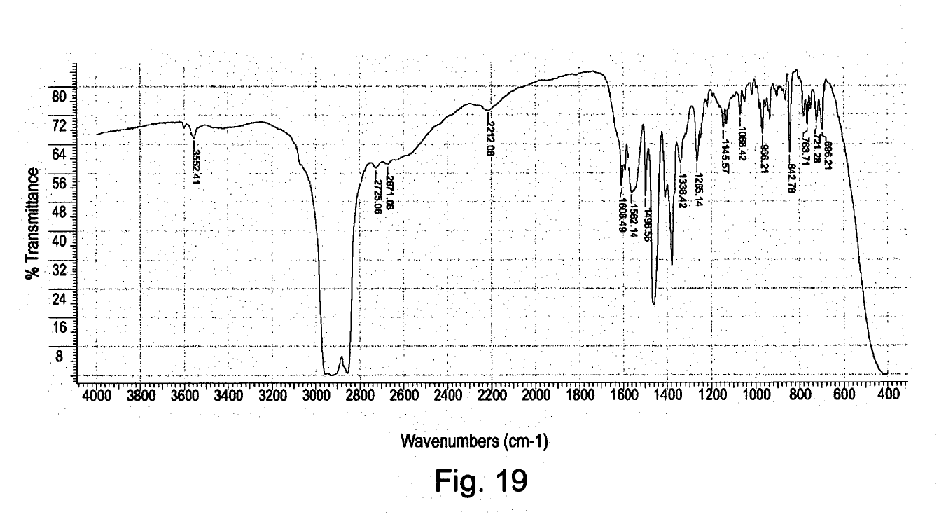

The montelukast cyclohexyl ammonium salt form V is further characterized by a unique infra-red spectrum, which is depicted in FIG. 19.

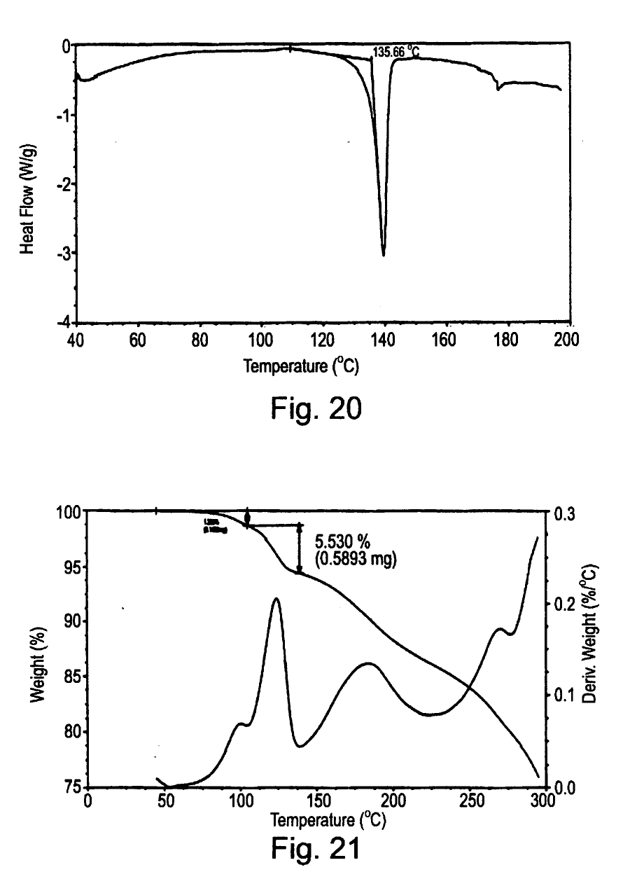

The montelukast cyclohexyl ammonium salt form V is further characterized by DSC and TGA curves, which are depicted in FIGS. 20 and 21 respectively.

In yet another embodiment, the present invention provides a process for preparing the crystalline solid comprising montelukast cyclohexyl ammonium salt form V, the process comprising:
providing a mixture of montelukast acid in ethyl acetate while stirring and optionally heating to elevated temperature;
adding cyclohexylamine and cyclohexane and optionally cooling to obtain a suspension;
stirring for sufficient time to allow crystallization;
obtaining the crystals by filtering and washing; and
optionally drying the obtained crystals.

Preferably, the solvent used for washing is a 1:1 mixture of ethyl acetate and cyclohexane.

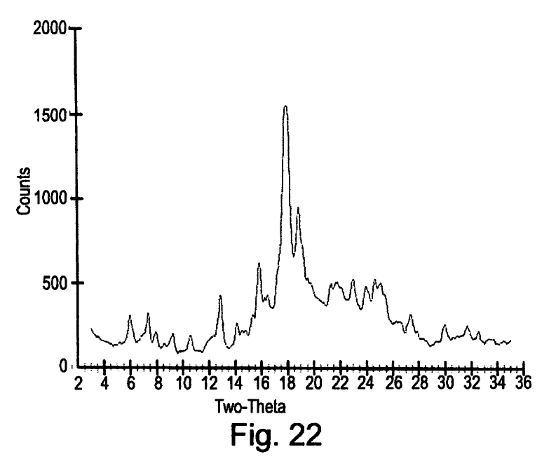

In yet another embodiment, the present invention provides a crystalline solid comprising montelukast phenethyl ammonium salt form VI. The montelukast phenethyl ammonium salt form VI is characterized by unique powder X-ray diffraction pattern (table 6, FIG. 22). The diffraction peaks at 15.9, 18.0, and 18.9±0.2 degrees 2θ are most characteristic of this form.

TABLE 6
montelukast phenethyl ammonium salt form VI - Powder X-ray
diffraction peak positions and intensities

| 2θ degrees | I/I0 |
|---|---|
| 6.0 | 11.4 |
| 7.0 | 4.3 |
| 7.4 | 13.4 |
| 8.0 | 5.9 |
| 8.6 | 1.8 |
| 9.2 | 5.7 |
| 10.6 | 5.7 |
| 12.9 | 18.1 |
| 14.1 | 7.7 |
| 14.5 | 4.0 |
| 15.4 | 8.6 |
| 15.9 | 25.2 |
| 16.3 | 14.2 |
| 16.5 | 14.8 |
| 18.0 | 100.0 |
| 18.9 | 41.7 |
| 19.6 | 17.7 |
| 21.3 | 16.8 |
| 21.8 | 16.8 |
| 23.1 | 17.6 |
| 24.0 | 15.6 |
| 24.7 | 17.9 |
| 25.1 | 16.6 |
| 25.4 | 12.8 |
| 26.6 | 5.1 |
| 27.3 | 8.8 |
| 30.0 | 6.4 |
| 31.7 | 6.8 |
| 32.6 | 3.8 |
| 33.4 | 2.0 |
| 33.8 | 2.8 |
| 34.4 | 1.6 |

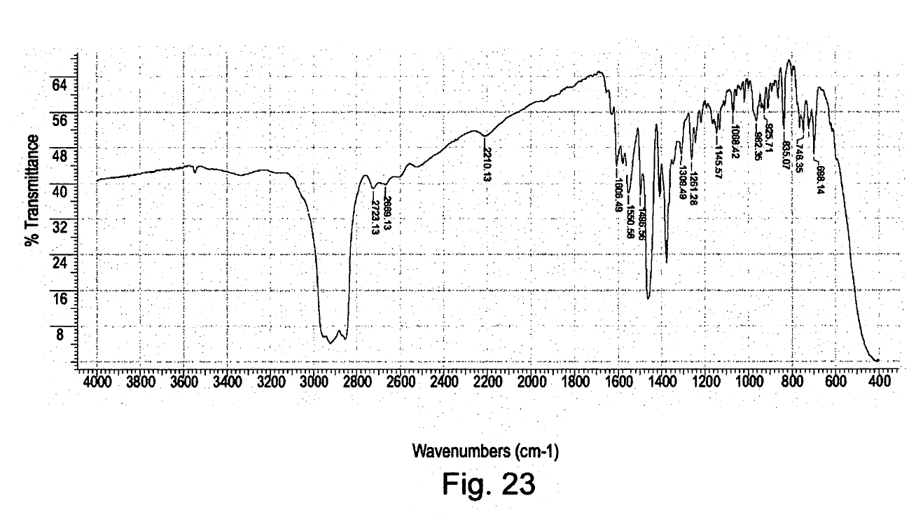

The montelukast phenethyl ammonium salt form VI is further characterized by a unique infra-red spectrum, which is depicted in FIG. 23.

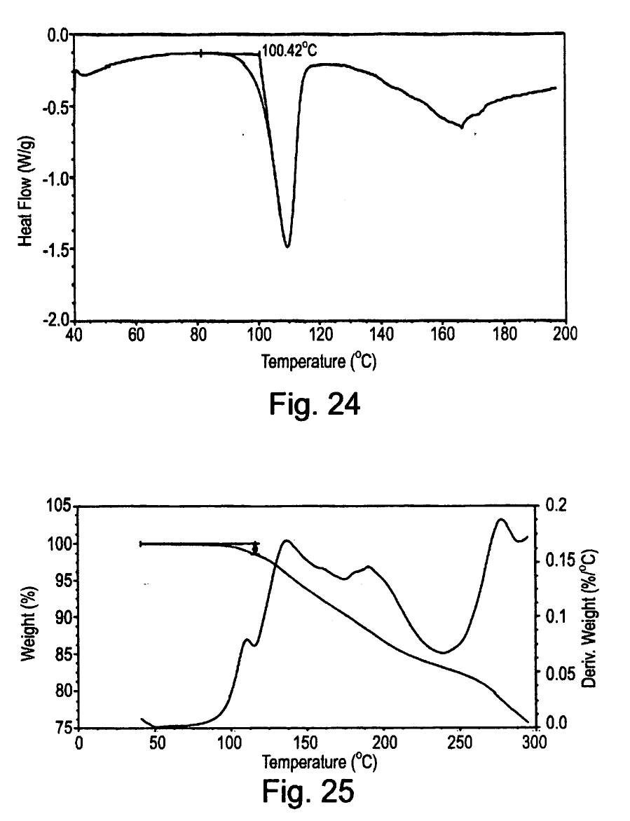

The montelukast phenethyl ammonium salt form VI is further characterized by DSC and TGA curves, which are depicted in FIGS. 24 and 25 respectively.

In yet another embodiment, the present invention provides a process for preparing the crystalline solid comprising montelukast phenethyl ammonium salt form VI, the process comprising:
providing a mixture of montelukast acid in ethyl acetate while stirring and optionally heating to elevated temperature;
adding phenetylamine and cyclohexane and optionally cooling to obtain a suspension;
stirring for sufficient time to allow crystallization;
obtaining the crystals by filtering and washing; and
optionally drying the obtained crystals.

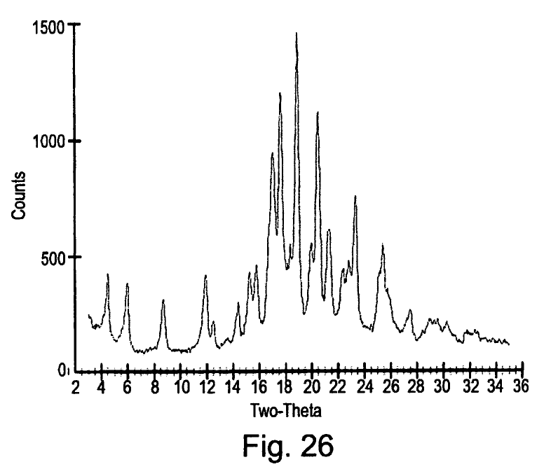

In yet another embodiment, the present invention provides a crystalline solid comprising montelukast cyclopentyl ammonium salt form VII. The montelukast cyclopentyl ammonium salt form VII is characterized by unique powder X-ray diffraction pattern (table 7, FIG. 26). The diffraction peaks at 4.5, 6.0, 11.9, 15.3, 15.8, 17.0, 17.6, 18.4, 18.9, 20.0, 20.5, 21.3, 22.4, 22.8, 23.3, 25.1 and 25.4±0.2 degrees 2θ are most characteristic of this form.

TABLE 7
montelukast cyclopentyl ammonium salt form VII - Powder X-ray
diffraction peak positions and intensities

| 2θ degrees | I/I0 |
|---|---|
| 4.5 | 21.6 |
| 6.0 | 22.3 |
| 8.7 | 18.4 |
| 11.9 | 24.2 |
| 12.5 | 7.8 |
| 14.4 | 13.9 |
| 15.3 | 22.0 |
| 15.8 | 24.3 |
| 17.0 | 58.5 |
| 17.6 | 79.0 |
| 18.4 | 29.4 |
| 18.9 | 100.0 |
| 20.0 | 29.6 |
| 20.5 | 72.6 |
| 21.3 | 33.1 |
| 22.4 | 21.4 |
| 22.8 | 24.0 |
| 23.3 | 46.8 |
| 25.1 | 21.4 |
| 25.4 | 30.1 |
| 25.7 | 16.2 |
| 27.5 | 9.6 |
| 28.9 | 7.4 |
| 29.5 | 8.1 |
| 30.2 | 7.0 |
| 31.8 | 5.4 |
| 32.4 | 4.9 |

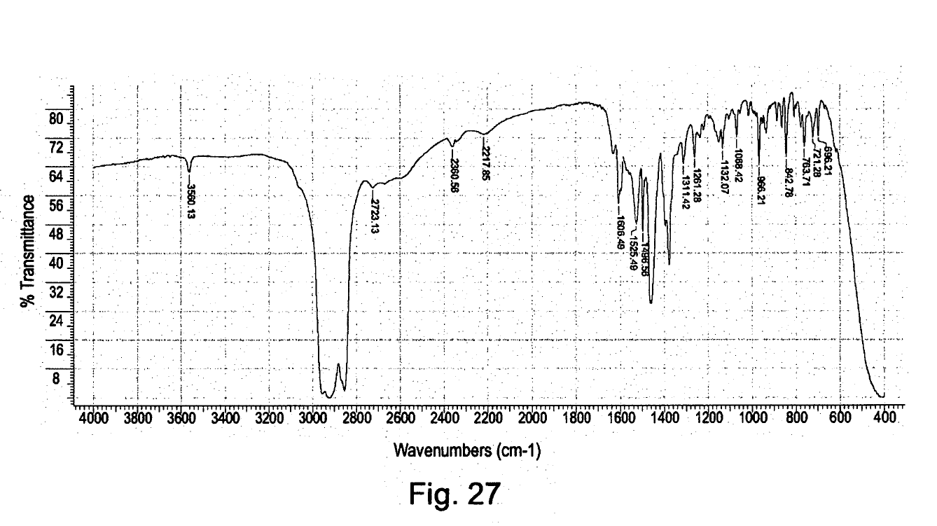

The montelukast cyclopentyl ammonium salt form VII is further characterized by a unique infra-red spectrum, which is depicted in FIG. 27.

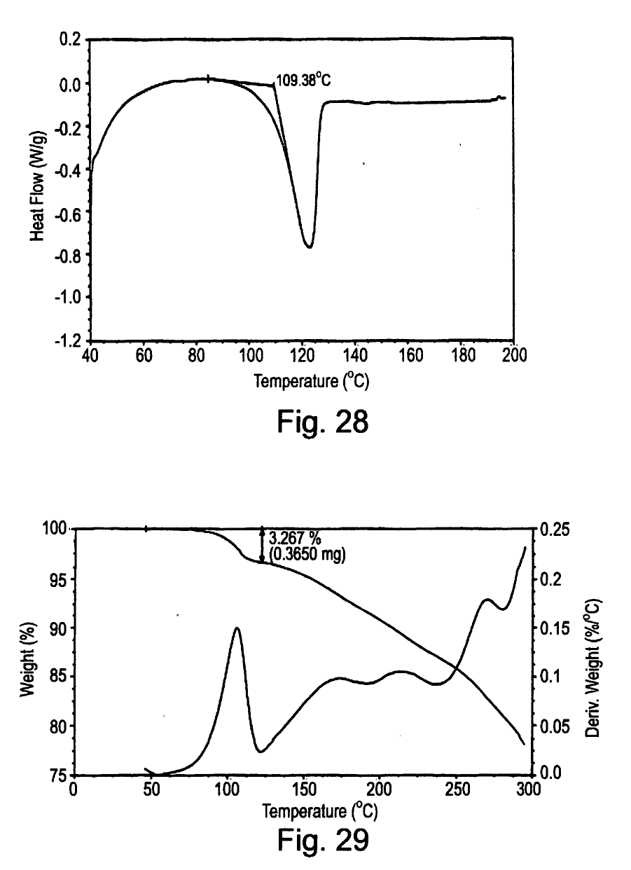

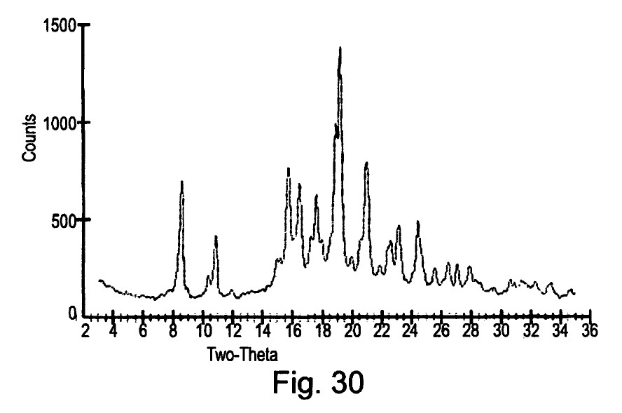

The montelukast cyclopentyl ammonium salt form VII is further characterized by DSC and TGA curves, which are depicted in FIGS. 28 and 29.

In yet another embodiment, the present invention provides a process for preparing the crystalline solid comprising montelukast cyclopentyl ammonium salt form VII, the process comprising:
providing a mixture of montelukast acid in acetonitrile while stirring and optionally heating to elevated temperature;
adding cyclopentylamine and optionally cooling to obtain a suspension;
stirring for sufficient time to allow crystallization;
obtaining the crystals by filtering and washing; and
optionally drying the obtained crystals.

In yet another embodiment, the present invention provides a crystalline solid comprising montelukast cyclooctyl ammonium salt form VIII. The montelukast cyclooctyl ammonium salt form VIII is characterized by unique powder X-ray diffraction pattern (table 8, FIG. 30). The strong diffraction peaks at 8.6, 10.9, 15.8, 16.5, 17.6, 19.0, 19.2, 21.0, 23.2 and 24.4±0.2 degrees 2θ are most characteristic of this form.

TABLE 8
montelukast cyclooctyl ammonium salt form VIII - Powder X-ray
diffraction peak positions and intensities

| 2θ degrees | I/I0 |
|---|---|
| 7.9 | 3.4 |
| 8.6 | 50.9 |
| 10.4 | 10.4 |
| 10.9 | 29.2 |
| 12.0 | 3.6 |
| 15.0 | 14.5 |
| 15.8 | 50.4 |
| 16.5 | 45.7 |
| 17.2 | 23.2 |
| 17.6 | 38.7 |
| 18.0 | 20.7 |
| 18.9 | 65.8 |
| 19.2 | 100.0 |
| 20.0 | 12.6 |
| 20.5 | 19.7 |
| 21.0 | 50.1 |
| 21.9 | 9.3 |
| 22.6 | 20.8 |
| 23.2 | 26.0 |
| 24.4 | 29.2 |
| 25.7 | 9.3 |
| 26.4 | 12.1 |
| 27.0 | 12.2 |
| 27.9 | 11.7 |
| 28.6 | 4.6 |
| 29.5 | 3.5 |
| 30.6 | 7.2 |
| 31.3 | 6.4 |
| 32.3 | 6.7 |
| 33.3 | 7.2 |
| 34.7 | 3.7 |

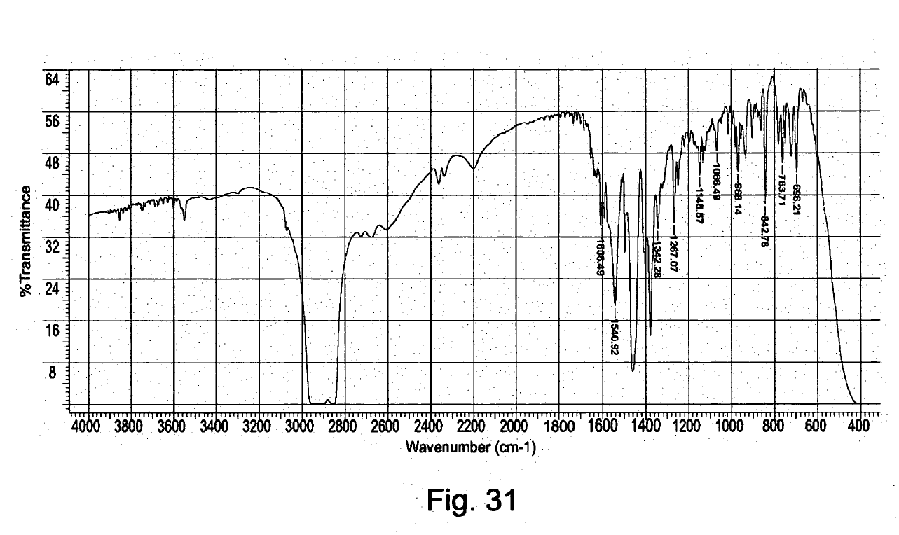

The montelukast cyclooctyl ammonium salt form VIII is further characterized by a characteristic IR spectrum as depicted in FIG. 31.

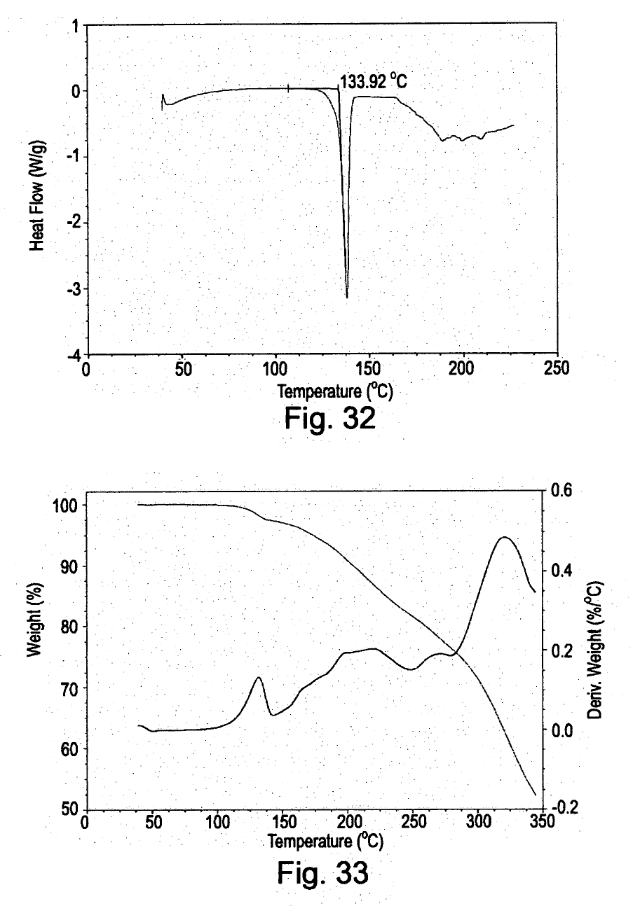

The montelukast cyclooctyl ammonium salt form VIII is further characterized by characteristic DSC and TGA curves as depicted in FIGS. 32 and 33 respectively.

In yet another embodiment, the present invention provides a process for preparing the crystalline solid comprising montelukast cyclooctyl ammonium salt form VIII, the process comprising:
providing a mixture of montelukast acid in an organic solvent while stirring and optionally heating to elevated temperature;
adding cyclooctylamine and optionally cooling to obtain a suspension;
stirring for sufficient time to allow crystallization;
obtaining the crystals by filtering and washing; and
optionally drying the obtained crystals.

Preferably, the organic solvent is selected from the group consisting of toluene, ethyl acetate, and acetonitrile.

In yet another embodiment, the present invention provides another process for preparing the crystalline solid comprising montelukast cyclooctyl ammonium salt form VIII, the process comprising:
providing a mixture of montelukast cyclooctyl ammonium salt in an organic solvent while stirring and heating to elevated temperature;
stirring for sufficient time to allow crystallization;
obtaining the crystals by filtering and washing; and
optionally drying the obtained crystals.

Preferably, the organic solvent is selected from the group consisting of toluene, diisopropyl ether, tetrahydrofuran (THF), ethyl acetate, acetone, methyl ethyl ketone (MEK), methanol, isopropanol, acetonitrile and mixtures thereof.

Yet another process of the present invention for preparing the crystalline solid comprising montelukast cyclooctyl ammonium salt form VIII, comprises:
providing a mixture of montelukast cyclooctyl ammonium salt in an organic solvent while stirring and heating to elevated temperature;
adding an anti-solvent upon cooling;
stirring for sufficient time to allow crystallization;
obtaining the crystals by filtering and washing; and
optionally drying the obtained crystals.

Preferably, the organic solvent is selected from the group consisting of cyclohexanone, dichloromethane, chloroform, toluene, m-xylene, 2-methoxyethyl ether, isobutyl acetate, t-butyl alcohol, n-amyl alcohol, benzyl alcohol, and mixtures thereof.

The anti-solvent is selected from the group consisting of n-hexane, cyclohexane, n-heptane, methyl t-butyl ether (MTBE), diisopropyl ether, ethoxymethyl ether, ethyl acetate, acetonitrile and mixtures thereof.

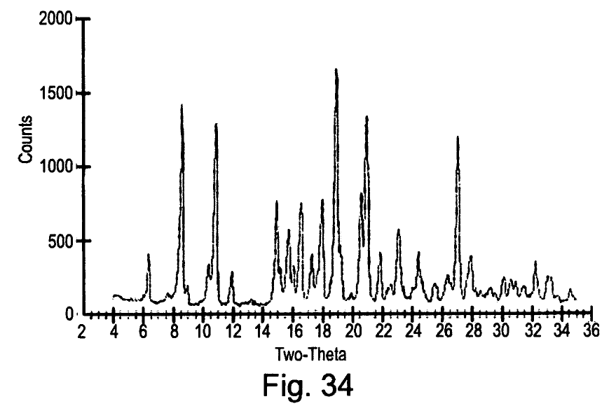

In yet another embodiment, the present invention provides a crystalline solid comprising montelukast cyclooctyl ammonium salt form IX. The montelukast cyclooctyl ammonium salt form IX is characterized by unique powder X-ray diffraction pattern (table 9, FIG. 34). The diffraction peaks at 8.6, 10.9, 14.9, 15.7, 16.5, 17.9, 18.9, 20.6, 20.9, 23.1 and 27.0±0.2 degrees 2θ are most characteristic of this form.

TABLE 9
montelukast cyclooctyl ammonium salt form IX - Powder X-ray
diffraction peak positions and intensities

| 2θ degrees | I/I0 |
|---|---|
| 6.4 | 21.5 |
| 7.6 | 4.7 |
| 8.6 | 86.7 |
| 9.0 | 8.0 |
| 10.4 | 18.2 |
| 10.9 | 83.8 |
| 11.9 | 15.8 |
| 13.2 | 3.6 |
| 14.9 | 47.3 |
| 15.2 | 15.6 |
| 15.7 | 32.6 |
| 16.1 | 16.1 |
| 16.5 | 43.5 |
| 17.3 | 22.6 |
| 17.9 | 44.8 |
| 18.9 | 100.0 |
| 19.9 | 4.6 |
| 20.6 | 49.3 |
| 20.9 | 78.8 |
| 21.9 | 20.8 |
| 22.6 | 7.6 |
| 23.1 | 32.0 |
| 24.1 | 5.2 |
| 24.4 | 21.5 |
| 25.5 | 7.4 |
| 26.4 | 11.1 |
| 27.0 | 70.8 |
| 27.9 | 18.7 |
| 28.6 | 4.0 |
| 29.2 | 5.3 |
| 30.1 | 9.9 |
| 30.6 | 8.7 |
| 30.9 | 8.1 |
| 31.4 | 5.9 |
| 32.2 | 16.7 |
| 33.0 | 10.1 |
| 34.6 | 4.7 |

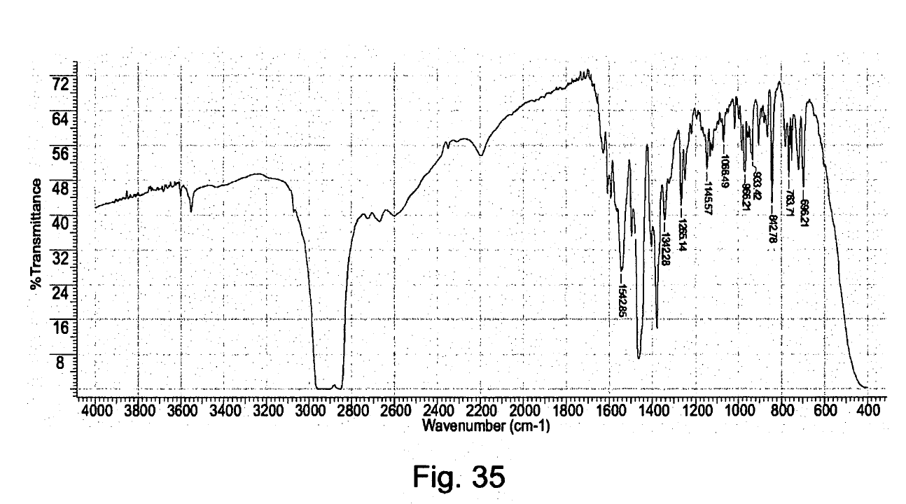

The montelukast cyclooctyl ammonium salt form IX, is further characterized by a unique infra-red spectrum, which is depicted in FIG. 35.

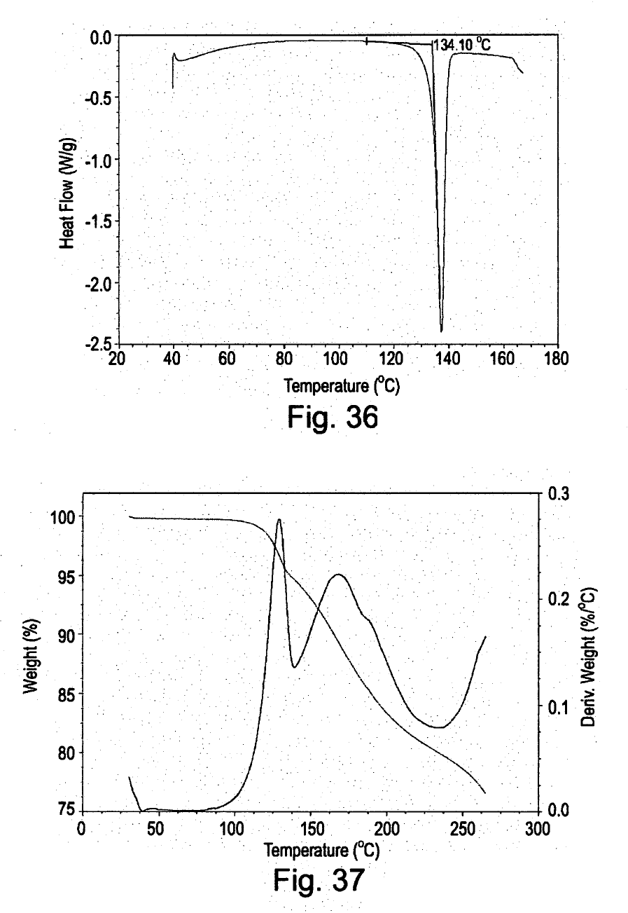

The montelukast cyclooctyl ammonium salt form IX, is further characterized by a DSC curve, which is depicted in FIG. 36.

The montelukast cyclooctyl ammonium salt form IX is further characterized by a thermogravimetric analysis (TGA) curve, which is depicted in FIG. 37.

In yet another embodiment, the present invention provides a process for preparing the montelukast cyclooctyl ammonium salt form IX, the process comprising:
providing a mixture of montelukast cyclooctyl ammonium salt in nitroethane while stirring and heating to elevated temperature;
stirring for sufficient time to allow crystallization;
obtaining the crystals by filtering and washing; and
optionally drying the obtained crystals.

Another process of the present invention for preparing the crystalline solid comprising montelukast cyclooctyl ammonium salt form IX, comprises:
providing a mixture of montelukast cyclooctyl ammonium salt in an organic solvent while stirring and heating to elevated temperature;
adding an anti-solvent upon cooling;
stirring for sufficient time to allow crystallization;
obtaining the crystals by filtering and washing; and
optionally drying the obtained crystals.

Preferably, the organic solvent is selected from N,N-dimethylformamide (DMF), chlorobenzene, and a mixture thereof.

Preferably, the antisolvent is methyl t-butylether (MTBE).

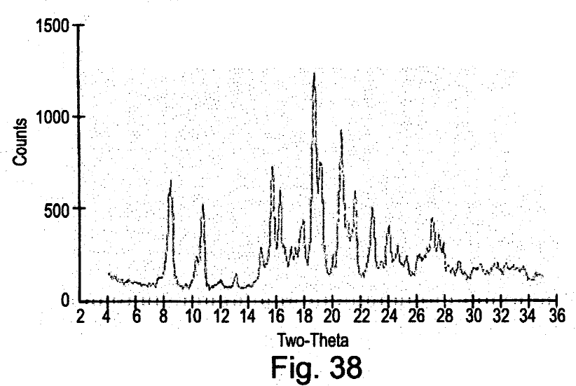

In yet another embodiment, the present invention provides a crystalline solid comprising montelukast cyclooctyl ammonium salt form X. The montelukast cyclooctyl ammonium salt form X is characterized by unique powder X-ray diffraction (table 10, FIG. 38). The diffraction peaks at 8.5, 10.8, 15.8, 16.3, 18.0, 18.8, 19.2, 20.7, 21.4, 21.0, 21.6, 22.9, 24.0, and 27.1±0.2 degrees 2θ are most characteristic of this form.

TABLE 10
montelukast cyclooctyl ammonium salt form X - Powder
X-ray diffraction peak positions and intensities

| 2θ degrees | I/I0 |
|---|---|
| 7.6 | 3.8 |
| 8.5 | 53.0 |
| 10.3 | 15.9 |
| 10.8 | 40.2 |
| 12.0 | 4.9 |
| 13.2 | 7.2 |
| 15.0 | 19.1 |
| 15.8 | 57.2 |
| 16.3 | 46.0 |
| 17.1 | 18.4 |
| 17.4 | 17.4 |
| 18.0 | 30.6 |
| 18.8 | 100.0 |
| 19.2 | 57.3 |
| 20.0 | 13.2 |
| 20.7 | 74.0 |
| 21.0 | 26.4 |
| 21.6 | 42.8 |
| 22.9 | 34.5 |
| 23.5 | 8.9 |
| 24.0 | 25.4 |
| 24.6 | 16.2 |
| 25.3 | 11.0 |
| 26.2 | 12.4 |
| 27.1 | 28.5 |
| 27.6 | 21.4 |
| 28.2 | 6.0 |
| 29.0 | 8.5 |
| 30.0 | 5.2 |
| 30.6 | 6.5 |
| 31.5 | 7.1 |
| 32.4 | 7.0 |
| 32.9 | 6.6 |
| 33.6 | 5.9 |

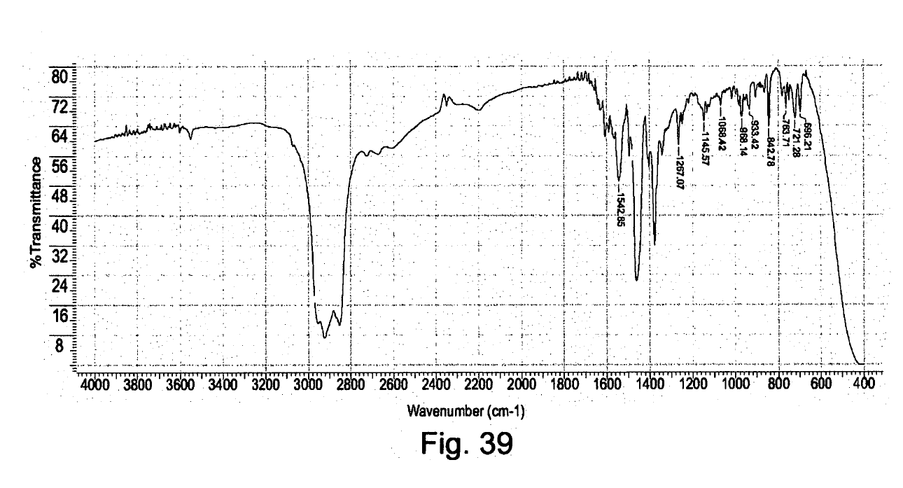

The montelukast cyclooctyl ammonium salt form X is further characterized by unique infra-red spectrum as depicted in FIG. 39.

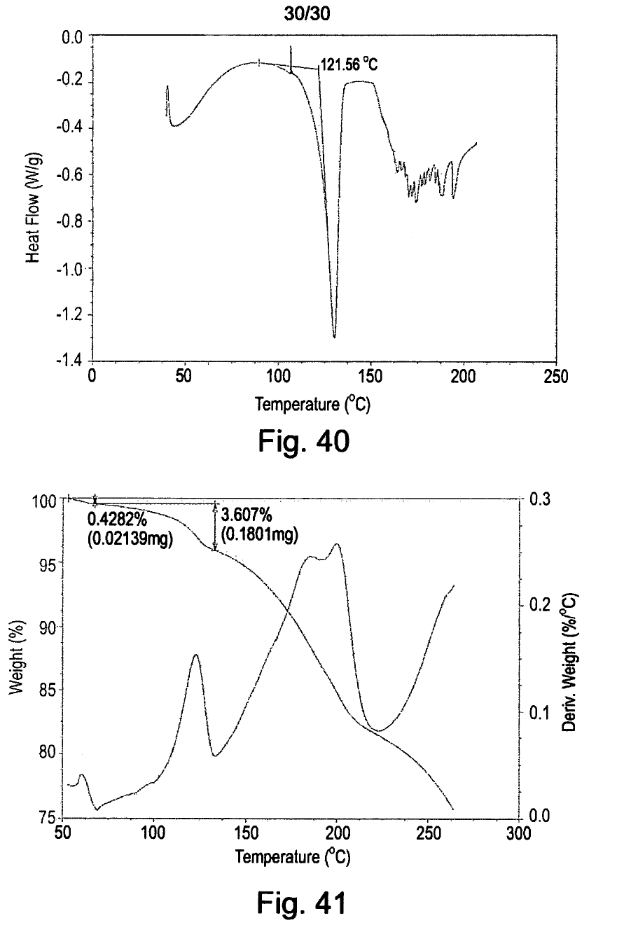

The montelukast cyclooctyl ammonium salt form X is further characterized by DSC and TGA curves as depicted in FIGS. 40 and 41 respectively.

In yet another embodiment, the present invention provides a process for preparing the crystalline solid comprising montelukast cyclooctyl ammonium salt form X, the process comprising:
providing a mixture of montelukast cyclooctyl ammonium salt in acetonitrile while stirring and heating to elevated temperature;
rapidly cooling the mixture while maintaining stirring;
obtaining the crystals by filtering and washing; and
optionally drying the obtained crystals.

Another process of the present invention for preparing the crystalline solid comprising montelukast cyclooctyl ammonium salt form X, comprises:
providing a mixture of montelukast cyclooctyl ammonium salt in an organic solvent while stirring and optionally heating to elevated temperature;
adding an anti-solvent upon cooling;
stirring for sufficient time to allow crystallization;
obtaining the crystals by filtering and washing; and
optionally drying the obtained crystals.

Preferably, the organic solvent is selected from chloroform, xylene and a mixture thereof.

Preferably, the anti-solvent is selected from n-heptane, diisopropyl ether methyl t-butyl ether (MTBE) and a mixture thereof.

In yet another embodiment, the present invention provides amorphous montelukast cyclooctyl ammonium salt.

### EXAMPLES

Reference is now made to the following examples, which together with the above descriptions, illustrate the invention in a non-limiting fashion. Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

The crystalline montelukast ammonium salts were characterized by powder X-ray diffraction, thereby generating fingerprint powder X-ray diffraction patterns for each particular crystalline form. Measurements of 2θ values typically are accurate to within±0.2 degrees 2θ. X-ray diffraction data were acquired using a PHILIPS X-ray diffractometer model PW1050-70. System description: Kα1=1.54178 Å, voltage 40 kV, current 28 mA, diversion slit=1°, receiving slit=0.2 mm, scattering slit=1° with a Graphite monochromator. Experiment parameters: pattern measured between 2θ=4° and 2θ=30° with 0.05° increments; count time was 0.5 second per increment.

The crystalline montelukast ammonium salts of the present invention were further characterized by Fourier-transform infrared (FTIR) spectroscopy to an accuracy of±4 cm⁻¹ using a Nicolet Fourier-Transform Infrared spectrometer model Avatar 360, with Omnic software version 5.2. All samples were run using KBr pellets.

FTIR is a well-known spectroscopic analytical tool, which measures the absorption of IR energy by a sample from transitions in molecular vibrational energy levels. While FTIR is primarily used for identification of functional groups in a molecule, different polymorphic forms also can exhibit differences in FTIR.

The crystalline montelukast cyclooctyl ammonium salts of the present invention also were characterized by differential scanning calorimetry (DSC), run on TA instruments model Q1000, with Universal software version 3.88. Samples were analyzed inside crimped 40 μl Aluminum pans. Heating rate for all samples was 5° C./min.

Differential scanning calorimetry (DSC) graphs were recorded using a TA Instruments Q1000 Thermal Analyzer with Universal software (version 3.88). Samples were analyzed inside crimped 40 μl Aluminum pans at a heating rate of 5° C./min.

The crystalline montelukast cyclooctyl ammonium salts of the present invention also were characterized by thermogravimetric analysis (TGA), a measure of the thermally induced weight loss of a material as a function of the applied temperature. Thermogravimetric analysis (TGA) was performed using a TA Instruments Q500 Thermal Analyzer with Universal Software (version 3.88). Samples were analyzed inside platinum baskets at a heating rate of 5° C./minute.

### Example 1

A 500 ml 3-necked flask equipped with a thermometer, a nitrogen inlet and a magnetic stirrer was charged at room temperature with 1.8 g (0.0123 moles) of 1-(mercaptomethyl)cyclopropaneacetic acid and 16 ml of DMF under stirring and under nitrogen atmosphere to obtain a solution. 1.8 ml of NaOH 47% (0.032 moles) was added drop-wise and stirring was maintained for 10 minutes to afford a suspension. A solution of 3 g of (S)-1-(3-(2-(7-chloro-2-quinolinyl)ethenyl(phenyl)-3(-2-(1-hydroxy-1-methylethyl)-phenyl)propyl methanesulfonate (0.0056 moles) in 20 ml THF was added in portions at 25° C. After completing the addition, the mixture was stirred for 2 hours at 25° C. and reaction completion was checked by HPLC. 43 ml of ethyl acetate was added to the reaction mixture and 43 ml of 5% sodium chloride solution. The mixture was stirred at 25° C. for 15 minutes.

Then, the layers were separated and 28 ml of 0.5 M tartaric acid was added to the upper layer and stirring was maintained at 25° C. for 15 minutes. The layers were separated and the upper layer was washed with 14 ml of water and again separated. The organic layer was distilled to dryness to afford an oily residue. 34 ml of ethyl acetate was added to the residue and the mixture was distilled to dryness to afford 3.8 g of an oily residue. 34 ml of ethyl acetate was added to the residue under stirring to obtain a solution. 1.44 g (0.0145 moles) of cyclohexylamine was added and stirring was maintained for few minutes at 25° C. and the solution was seeded with crystalline montelukast acid cyclohexyl ammonium salt. Stirring was maintained at 25° C. to afford a suspension, which was filtered to obtain a cake. The cake was washed with ethyl acetate and dried at 40° C. in vacuum to afford 2.9 g of dry crude montelukast acid cyclohexyl ammonium salt in 70% yield. The purity of the crude product was 99% (by HPLC).

### Example 2

A reaction vessel equipped with a thermometer, a reflux condenser and a magnetic stirrer was charged with 3 g (0.0051 mol) of montelukast acid in 45 ml of ethyl acetate. The mixture was stirred and heated to a temperature of about 60° C. to afford a solution. 0.595 g (0.0060 moles) of cyclohexylamine was added followed by addition of 45 ml of cyclohexane, and the mixture was cooled to 25° C., during which time a suspension was formed. Stirring was maintained for 1 hour at 25° C. The thus formed crystals were filtered, washed with cold mixture of 1:1 ethyl acetate: cyclohexane and dried under vacuum at 40° C. to obtain 3.04 g (87% yield) of the product, which was characterized as crystalline montelukast cyclohexyl ammonium salt form V, having a melting point of 137.7-140° C., and a purity of 98.8% (by HPLC).

### Example 3

In a similar manner, montelukast phenethyl ammonium salt form VI was prepared (using phenethyl amine instead of cyclohexylamine) in 81% yield, having a melting point of 116.9-118.9° C., and a purity of 97% (by HPLC).

### Example 4

A reaction vessel equipped with a thermometer, a reflux condenser and a magnetic stirrer was charged with 3 g (0.0051 moles) of montelukast acid in 45 ml of ethyl acetate. The mixture was stirred and heated to a temperature of about 60° C. to afford a solution. 0.679 g (0.0060 moles) of cycloheptylamine was added, and the mixture was cooled to 25° C., causing the montelukast cycloheptyl ammonium salt to separate and the resulting suspension was stirred for 1 hour at 25° C. The thus formed crystals were filtered, washed with cold ethyl acetate and dried under vacuum at 40° C. to obtain 3.39 g (95% yield) of the product, which was characterized as crystalline montelukast cycloheptyl ammonium salt form II, having a purity of 99.1% (by HPLC).

### Examples 5-8

Other montelukast ammonium salts were prepared in a similar procedure to the one described in Example 4 and as detailed in Table 11.

TABLE 11
Crystallization experiments from ethyl acetate

| | | Poly- | | | Purity |
|---|---|---|---|---|---|
| Exam- | | morph | | | % |
| ple | | form | Melting | | (by |
| No. | The amine used | No. | point | Yield | HPLC) |
| 5 | phenethylamine | I | 66.2-67.9° C. | 77% | 99.4 |
| 6 | cyclopentylamine | III | 126.9-128.8 | 62% | 98.5 |
| 7 | cyclooctylamine | VIII | 136.6-138.9 | 92% | 99.2 |
| 8 | cyclododecylamine | IV | 139.3-140 | 95% | 99.0 |

### Example 9

A reaction vessel equipped with a thermometer, a reflux condenser and a magnetic stirrer was charged with 3 g (0.0051 moles) of montelukast acid in 45 ml of acetonitrile. The mixture was stirred and heated to reflux to afford a suspension. 0.511 g (0.0060 moles) of cyclopentylamine was added, and the mixture was cooled to 25° C., causing the montelukast cyclopentyl ammonium salt to separate and the resulting suspension was stirred for 1 hour at 25° C. The thus formed crystals were filtered, washed with cold acetonitrile and dried under vacuum at 40° C. to obtain 3.25 g (95% yield) of crystalline montelukast cyclopentyl ammonium salt form VII, having a melting point of 126.3-128° C., and a purity of 98.8% (by HPLC).

### Examples 10-13

Other montelukast ammonium salts were prepared in a similar procedure to the one described in Example 9 and as detailed in Table 12.

TABLE 12
Crystallization experiments from acetonitrile

| Example | | Polymorph | | Purity % |
|---|---|---|---|---|
| No. | The amine used | form No. | Yield | (by HPLC) |
| 10 | cyclohexylamine | II | 96% | 99.1 |
| 11 | cycloheptylamine | II | 95% | 99.0 |
| 12 | cyclooctylamine | VIII | 95% | 99.0 |
| 13 | cyclododecylamine | IV | 89% | 98.9 |

### Example 14

A reaction vessel equipped with a thermometer, a reflux condenser and a magnetic stirrer was charged with 3 g (0.0051 moles) of montelukast acid in 45 ml of toluene. The mixture was stirred and heated to reflux to afford a solution. 0.763 g (0.0060 moles) of cyclooctylamine was added, and the mixture was cooled to 25° C., causing the montelukast cyclooctyl ammonium salt to separate and the resulting suspension was stirred for 1 hour at 25° C. The thus formed crystals were filtered, washed with cold toluene and dried under vacuum at 40° C. to obtain 3.42 g (94% yield) of crystalline montelukast cyclooctyl ammonium salt form VIII, having a purity of 98.5% (by HPLC).

### Examples 15-16

Other montelukast ammonium salts were prepared in a similar procedure to the one described in Example 14 and as detailed in Table 13.

TABLE 13
Crystallization experiments from toluene

| Exam- | | Poly- | | | Purity % |
|---|---|---|---|---|---|
| ple | | morph | Melting | | (by |
| No. | The amine used | form No. | point | Yield | HPLC) |
| 15 | cycloheptylamine | II | 137.5-138.5 | 95% | 97.7 |
| 16 | cyclopentylamine | III | 126.8-128.3 | 70% | 98.0 |

### Example 17

A reaction vessel equipped with a thermometer, a reflux condenser and a magnetic stirrer was charged with 3 g (0.0051 mol) of montelukast acid in 45 ml of toluene. The mixture was stirred and heated to reflux to afford a solution. 1.1 g (0.0060 mol) of cyclododecylamine was added, followed by addition of 45 ml of cyclohexane, and the mixture was cooled to 25° C., causing the montelukast cyclododecyl ammonium salt to separate and the resulting suspension was stirred for 1 hour at 25° C. The thus formed crystals were filtered, washed with a 1:1 mixture of toluene and cyclohexane, and dried under vacuum at 40° C. to obtain 3.73 g (95% yield) of crystalline montelukast cyclododecyl ammonium salt form IV, having a purity of 98.0% (by HPLC).

### Example 18

In a similar procedure to the one described in Example 17, montelukast cyclohexyl ammonium salt form II was prepared (using cyclohexylamine instead of cyclododecylamine) in about 100% yield, having a purity of 97.2% (by HPLC).

### Example 19

A reaction vessel equipped with a thermometer, a reflux condenser and a magnetic stirrer was charged with 0.56 g of crude montelukast cyclooctyl ammonium salt in 53 ml of acetone. The mixture was stirred and heated to reflux to afford a solution, and left to cool for a sufficient time period to allow crystallization. The thus formed crystals were filtered, washed with cold acetone and dried under vacuum to obtain crystalline montelukast cyclooctyl ammonium salt form VIII, having a purity of 97.5%.

### Examples 20-25

Other preparations of montelukast cyclooctyl ammonium salt form VIII have been carried out in a similar manner to the procedure described in example 19, and as detailed in Table 14.

TABLE 14
Crystallization experiments from different solvents

| Example | | Solid:solvent | Yield | |
|---|---|---|---|---|
| No. | Solvent | ratio, g:ml | % | Purity % |
| 20 | toluene | 0.5:5 | 80 | ND |
| 21 | diisopropyl ether:methanol | 0.5:78 | ND | ND |
| | 25:1 (v/v) | | | |
| 22 | acetonitrile:toluene | 0.5:12.5 | ND | ND |
| | 4:1 (v/v) | | | |
| 23 | THF:ethyl acetate | 0.6:350 | 50% | ND |
| | 69:1 (v/v) | | | |
| 24 | methyl ethyl ketone | 0.5:26 | 36% | 91.8 |
| | (MEK) | | | |
| 25 | isopropanol | 0.5:9 | 98 | 97.6 |

ND = not determined

### Example 26

A reaction vessel equipped with a thermometer, a reflux condenser and a magnetic stirrer was charged with 0.5 g of crude montelukast cyclooctyl ammonium salt in 4 ml of n-octanol. The mixture was stirred and heated to 100° C. to afford a solution, which was evaporated to dryness under vacuum to obtain amorphous montelukast cyclooctyl ammonium salt.

### Example 27

A reaction vessel equipped with a thermometer, a reflux condenser and a magnetic stirrer was charged with 0.76 g of crude montelukast cyclooctyl ammonium salt in 7 ml of isobuyl acetate. The mixture was stirred and heated to reflux to afford a solution, and 5 ml of diisopropyl ether was added drop-wise upon cooling. The thus formed crystals were filtered, washed with cold diisopropyl ether and dried under vacuum to obtain 0.6 g (79% yield) of the product, which was characterized as crystalline montelukast cyclooctyl ammonium salt form VIII, having a purity of 97.6%.

### Examples 28-37

Other preparations of montelukast cyclooctyl ammonium salts form VIII have been carried out in a similar manner to the procedure described in example 27, and as detailed in Table 15.

TABLE 15
Crystallization experiments from different mixtures of solvents and
anti-solvents

| | | | | Solid:solvent:anti- | |
|---|---|---|---|---|---|
| Example | | | Heating | solvent | |
| No. | Solvent | Anti-solvent | temperature ° C. | ratio g:ml:ml | Yield % |
| 28 | dichloromethane | diisopropyl ether | reflux | 0.5:10:25 | 99.9 |
| 29 | benzyl alcohol | diisopropyl ether | ambient | 0.7:2:20 | 71.4 |
| | | | temperature | | |
| 30 | benzyl alcohol | ethyl acetate | ambient | 0.7:2:12 | ND |
| | | | temperature | | |
| 31 | cyclohexanone | hexane | 100 | 0.5:4:7 | 90 |
| 32 | 2-methoxyethyl | acetonitrile | reflux | 0.54:30:18 | 74.1 |
| | ether | | | | |
| 33 | chloroform | cyclohexane | reflux | 0.48:2:100 | 99.9 |
| 34 | t-butyl alcohol | diisopropyl ether | reflux | 0.54:8:16 | 64.8 |
| 35 | toluene | acetonitrile | 108 | 0.55:5:4 | 65.5 |
| 36 | m-xylene | cyclohexane | reflux | 0.64:7.5:7.5 | 70.3 |
| 37 | n-amyl alcohol | ethoxymethyl | 90 | 0.45:3:4 | 82.2 |
| | | ether | | | |

ND = not determined

### Example 38

A reaction vessel equipped with a thermometer, a reflux condenser and a magnetic stirrer was charged with 0.7 g of crude montelukast cyclooctyl ammonium salt in 77 ml of nitroethane. The mixture was stirred and heated to 105° C. to afford a solution. The thus formed crystals were filtered and dried under vacuum to obtain 0.63 g (90% yield) of the product, which was characterized as crystalline montelukast cyclooctyl ammonium salt form IX.

### Example 39

A reaction vessel equipped with a thermometer, a reflux condenser and a magnetic stirrer was charged with 0.5 g of crude montelukast cyclooctyl ammonium salt in 5 ml of a 1:4 mixture (v/v) of DMF:chlorobenzene. The mixture was stirred and heated to a temperature of 66° C. to afford a solution, and 13 ml of methyl t-butyl ether (MTBE) was added drop-wise upon cooling. The thus formed crystals were filtered, washed with cold MTBE and dried under vacuum to obtain 0.4 g (80% yield) of the product, which was characterized as crystalline montelukast cyclooctyl ammonium salt form IX, having a purity of 97.6%.

### Example 40

A reaction vessel equipped with a thermometer, a reflux condenser and a magnetic stirrer was charged with 0.6 g of crude montelukast cyclooctyl ammonium salt in 107 ml of acetonitrile. The mixture was stirred and heated to a temperature of about 40° C. for about two hours, after which time the mixture was rapidly cooled to about 0° C. maintaining intensive mixing. The thus formed crystals were filtered, washed with cold acetonitrile and dried under vacuum to obtain crystalline montelukast cyclooctyl ammonium salt form X, having a purity of 97.5%.

### Example 41

A reaction vessel equipped with a thermometer, a reflux condenser and a magnetic stirrer was charged with 0.56 g of crude montelukast cyclooctyl ammonium salt in 30 ml of a 5:1 mixture of chloroform and xylene (v/v). The mixture was stirred at ambient temperature, and 12 ml of a mixture of 9.5:2.5 diisopropyl ether:n-heptane (v/v) was added drop-wise upon cooling. The thus formed crystals were filtered, washed with the cold mixture of diisopropyl ether and n-heptane and dried under vacuum to obtain crystalline montelukast cyclooctyl ammonium salt form X, having a purity of 81.1%.

### Example 42

A reaction vessel equipped with a thermometer, a reflux condenser and a magnetic stirrer was charged with 0.5 g of crude montelukast cyclooctyl ammonium salt in 35 ml of chloroform. The mixture was stirred and heated to reflux to afford a solution, and 15 ml of methyl t-butyl ether (MTBE) was added drop-wise upon cooling. The thus formed crystals were filtered, washed with cold MTBE and dried under vacuum to obtain crystalline montelukast cyclooctyl ammonium salt form X in 80% yield.

What is claimed is:

1. An ammonium addition salt of R-(E)-1-(((1-(3-(2-(7-chloro-2-quinolinyl)-ethenyl)phenyl)-3-(2-(1-hydroxy-1-methylethyl)phenyl)propyl)thio)methyl)-cyclopropaneacetic acid (montelukast acid) selected from the group consisting of cyclopentyl ammonium salt, cyclohexyl ammonium salt, cycloheptyl ammonium salt, cyclododecyl ammonium salt, phenethyl ammonium salt, and cyclooctyl ammonium salt.

2. A crystalline solid comprising montelukast ammonium salt selected from the group consisting of cyclopentyl ammonium salt, cyclohexyl ammonium salt, cycloheptyl ammonium salt, cyclododecyl ammonium salt, phenethyl ammonium salt, and cyclooctyl ammonium salt.

3. The crystalline solid comprising montelukast phenethyl ammonium salt form I of claim 2, characterized by powder X-ray diffraction pattern, wherein the strong diffraction peaks at 8.0, 15.3, 16.5, 17.3, 18.1, 20.7, 21.3, 22.4, 24.4, and 25.±0.2 degrees 2θ are most characteristic of this form.

4. A process for preparing the crystalline solid comprising montelukast phenethyl ammonium salt form I, the process comprising:
providing a mixture of montelukast acid in ethyl acetate while stirring and optionally heating to elevated temperature;
adding phenethylamine and optionally cooling to obtain a suspension;
stirring for sufficient time to allow crystallization;
obtaining the crystals by filtering and washing; and
optionally drying the obtained crystals.

5. The crystalline solid comprising montelukast cyclohexyl ammonium salt form II, or montelukast cycloheptyl ammonium salt form II of claim 2, characterized by powder X-ray diffraction pattern, wherein the diffraction peaks at 8.8, 10.7, 15.7, 16.4, 16.6, 17.7, 19.4, and 21.4±0.2 degrees 2θ are most characteristic of this form.

6. A process for preparing the crystalline solid comprising montelukast cyclohexyl ammonium salt form II, the process comprising:
providing a mixture of montelukast acid in an organic solvent while stirring and optionally heating to elevated temperature;
adding cyclohexylamine and optionally cooling to obtain a suspension;
stirring for sufficient time to allow crystallization;
obtaining the crystals by filtering and washing; and
optionally drying the obtained crystals.

7. The process of claim 6, wherein the organic solvent is selected from the group consisting of cyclohexane, toluene, ethyl acetate, acetonitrile, and mixtures thereof.

8. A process for preparing the crystalline solid comprising montelukast cycloheptyl ammonium salt form II, the process comprising:
providing a mixture of montelukast acid in an organic solvent while stirring and optionally heating to elevated temperature;
adding cycloheptylamine and optionally cooling to obtain a suspension;
stirring for sufficient time to allow crystallization;
obtaining the crystals by filtering and washing; and
optionally drying the obtained crystals.

9. The process of claim 8, wherein the organic solvent is selected from the group consisting of toluene, ethyl acetate, acetonitrile, and mixtures thereof.

10. The crystalline solid comprising montelukast cyclopentyl ammonium salt form III of claim 2, characterized by powder X-ray diffraction, wherein the diffraction peaks at 9.2, 11.1, 15.5, 16.0, 16.2, 17.0, 17.6, 18.5, 19.3, 20.3, 20.9, 21.4, 21.7, 22.2, 23.3, 24.7, and 25.2±0.2 degrees 2θ are most characteristic of this form.

11. A process for preparing the crystalline solid comprising montelukast cyclopentyl ammonium salt form III, the process comprising:
providing a mixture of montelukast acid in an organic solvent while stirring and optionally heating to elevated temperature;
adding cyclopentylamine and optionally cooling to obtain a suspension;
stirring for sufficient time to allow crystallization;
obtaining the crystals by filtering and washing; and
optionally drying the obtained crystals.

12. The process of claim 11, wherein the organic solvent is selected from the group consisting of toluene and ethyl acetate.

13. The crystalline solid comprising montelukast cyclododecyl ammonium salt form IV of claim 2, characterized by unique powder X-ray diffraction pattern, wherein the diffraction peaks at 7.7, 10.5, 13.0, 14.0, 17.7, 18.4, 19.7, 21.5, 21.9, 23.8, 25.2 and 27.4 2±0.2 degrees 2θ are most characteristic of this form.

14. A process for preparing the crystalline solid comprising montelukast cyclododecyl ammonium salt form IV, the process comprising:
providing a mixture of montelukast acid in an organic solvent while stirring and optionally heating to elevated temperature;
adding cyclododecylamine and optionally cooling to obtain a suspension;
stirring for sufficient time to allow crystallization;
obtaining the crystals by filtering and washing; and
optionally drying the obtained crystals.

15. The process of claim 14, wherein the organic solvent is selected from the group consisting of cyclohexane, toluene, ethyl acetate, acetonitrile, and mixtures thereof.

16. The crystalline solid comprising montelukast cyclohexyl ammonium salt form V, of claim 2, characterized by powder X-ray diffraction pattern, wherein, the diffraction peaks at 4.5, 8.3, 8.7, 9.8, 10.8, 15.7, 16.2, 16.7, 17.8, 18.4, 19.7, 21.2, 21.5, 22.6, 23.1, 23.4, 24.0 25.5, and 27.0±0.2 degrees 2θ are most characteristic of this form.

17. A process for preparing the crystalline solid comprising montelukast cyclohexyl ammonium salt form V, the process comprising:
providing a mixture of montelukast acid in ethyl acetate while stirring and optionally heating to elevated temperature;
adding cyclohexylamine and cyclohexane and optionally cooling to obtain a suspension;
stirring for sufficient time to allow crystallization;
obtaining the crystals by filtering and washing; and
optionally drying the obtained crystals.

18. The crystalline solid comprising montelukast phenethyl ammonium salt form VI, of claim 2, characterized by powder X-ray diffraction pattern, wherein the diffraction peaks at 15.9, 18.0, and 18.9±0.2 degrees 2θ are most characteristic of this form.

19. A process for preparing the crystalline solid comprising montelukast phenethyl ammonium salt form VI, the process comprising:
providing a mixture of montelukast acid in ethyl acetate while stirring and optionally heating to elevated temperature;
adding phenetylamine and cyclohexane and optionally cooling to obtain a suspension;
stirring for sufficient time to allow crystallization;
obtaining the crystals by filtering and washing; and
optionally drying the obtained crystals.

20. The crystalline solid comprising montelukast cyclopentyl ammonium salt form VII of claim 2, characterized by powder X-ray diffraction pattern, wherein the diffraction peaks at 4.5, 6.0, 11.9, 15.3, 15.8, 17.0, 17.6, 18.4, 18.9, 20.0, 20.5, 21.3, 22.4, 22.8, 23.3, 25.1 and 25.4±0.2 degrees 2θ are most characteristic of this form.

21. A process for preparing the crystalline solid comprising montelukast cyclopentyl ammonium salt form VII, the process comprising:
providing a mixture of montelukast acid in acetonitrile while stirring and optionally heating to elevated temperature;
adding cyclopentylamine and optionally cooling to obtain a suspension;
stirring for sufficient time to allow crystallization;
obtaining the crystals by filtering and washing; and
optionally drying the obtained crystals.

22. The crystalline solid comprising montelukast cyclooctyl ammonium salt form VIII of claim 2, characterized by powder X-ray diffraction pattern, wherein the diffraction peaks at 8.6, 10.9, 15.8, 16.5, 17.6, 19.0, 19.2, 21.0, 23.2 and 24.4±0.2 degrees 2θ are most characteristic of this form.

23. A process for preparing the crystalline solid comprising montelukast cyclooctyl ammonium salt form VIII, the process comprising:
providing a mixture of montelukast acid in an organic solvent while stirring and optionally heating to elevated temperature;
adding cyclooctylamine and optionally cooling to obtain a suspension;
stirring for sufficient time to allow crystallization;
obtaining the crystals by filtering and washing; and
optionally drying the obtained crystals.

24. The process according to claim 23, wherein the organic solvent is selected from the group consisting of toluene, ethyl acetate, and acetonitrile.

25. A process for preparing the crystalline solid comprising montelukast cyclooctyl ammonium salt form VIII, the process comprising:
providing a mixture of montelukast cyclooctyl ammonium salt in an organic solvent while stirring and heating to elevated temperature;
stirring for sufficient time to allow crystallization;
obtaining the crystals by filtering and washing; and
optionally drying the obtained crystals.

26. The process of claim 25, wherein the organic solvent is selected from the group consisting of toluene, diisopropyl ether, tetrahydrofuran (THF), ethyl acetate, acetone, methyl ethyl ketone (MEK), methanol, isopropanol, acetonitrile, and mixtures thereof.

27. A process for preparing the crystalline solid comprising montelukast cyclooctyl ammonium salt form VIII, The process comprising:
providing a mixture of montelukast cyclooctyl ammonium salt in an organic solvent while stirring and heating to elevated temperature;
adding an anti-solvent upon cooling;
stirring for sufficient time to allow crystallization;
obtaining the crystals by filtering and washing; and
optionally drying the obtained crystals.

28. The process of claim 27, wherein the organic solvent is selected from the group consisting of cyclohexanone, dichloromethane, chloroform, toluene, m-xylene, 2-methoxyethyl ether, isobutyl acetate, t-butyl alcohol, n-amyl alcohol, benzyl alcohol, and mixtures thereof.

29. The process of claim 27, wherein the anti-solvent is selected from the group consisting of n-hexane, cyclohexane, n-heptane, methyl t-butyl ether (MTBE), diisopropyl ether, ethoxymethyl ether, ethyl acetate, acetonitrile, and mixtures thereof.

30. The crystalline solid comprising montelukast cyclooctyl ammonium salt form IX of claim 2, characterized by unique powder X-ray diffraction pattern, wherein the diffraction peaks at 8.6, 10.9, 14.9, 15.7, 16.5, 17.9, 18.9, 20.6, 20.9, 23.1 and 27.0±0.2 degrees 2θ are most characteristic of this form.

31. A process for preparing the crystalline solid comprising montelukast cyclooctyl ammonium salt form IX, the process comprising:
providing a mixture of montelukast cyclooctyl ammonium salt in nitroethane while stirring and heating to elevated temperature;
stirring for sufficient time to allow crystallization;
obtaining the crystals by filtering and washing; and
optionally drying the obtained crystals.

32. A process for preparing the crystalline solid comprising montelukast cyclooctyl ammonium salt form IX, the process comprising:
providing a mixture of montelukast cyclooctyl ammonium salt in an organic solvent while stirring and heating to elevated temperature;
adding an anti-solvent upon cooling;
stirring for sufficient time to allow crystallization;
obtaining the crystals by filtering and washing; and
optionally drying the obtained crystals.

33. The process of claim 32, wherein the organic solvent is selected from N,N-dimethylformamide (DMF), chlorobenzene, and a mixture thereof.

34. The process of claim 32, wherein the anti-solvent is methyl t-butylether (MTBE).

35. The crystalline solid comprising montelukast cyclooctyl ammonium salt form X of claim 2, characterized by unique powder X-ray diffraction, wherein the diffraction peaks at 8.5, 10.8, 15.8, 16.3, 18.0, 18.8, 19.2, 20.7, 21.4, 21.0, 21.6, 22.9, 24.0, and 27.1±0.2 degrees 2θ are most characteristic of this form.

36. A process for preparing the crystalline solid comprising montelukast cyclooctyl ammonium salt form X, the process comprising:
providing a mixture of montelukast cyclooctyl ammonium salt in acetonitrile while stirring and heating to elevated temperature;
rapidly cooling the mixture while maintaining stirring;
obtaining the crystals by filtering and washing; and
optionally drying the obtained crystals.

37. A process for preparing the crystalline solid comprising montelukast cyclooctyl ammonium salt form X, the process comprising:
providing a mixture of montelukast cyclooctyl ammonium salt in an organic solvent while stirring and optionally heating to elevated temperature;
stirring for sufficient time to allow crystallization;
adding an anti-solvent upon cooling;
obtaining the crystals by filtering and washing; and
optionally drying the obtained crystals.

38. The process of claim 37, wherein the organic solvent is selected from chloroform, xylene and a mixture thereof.

39. The process of claim 37, wherein the anti-solvent is selected from n-heptane, diisopropyl ether, methyl t-butyl ether and a mixture thereof.

40. Amorphous montelukast cyclooctyl ammonium salt.

41. A process of claim 2 for preparing montelukast sodium using a montelukast ammonium salt selected from the group consisting of cyclopentyl ammonium salt, cyclohexyl ammonium salt, cycloheptyl ammonium salt, cyclododecyl ammonium salt, dicyclohexyl ammonium salt, phenethyl ammonium salt, and cyclooctyl ammonium salt.
